(19) United States
(12) Patent Application Publication
(10) Pub. No.: US 2008/0234333 A1
(43) Pub. Date: Sep. 25, 2008

(54) Novel Hydroxamic Acid Derivative as Peptide Deformylase Inhibitor and Manufacturing Method Thereof

(76) Inventors: Jae-Hoon Kang, Seoul (KR); Seung-Woo Yu, Seoul (KR); Hee-Yeol Lee, Seoul (KR); Kyung-Mi An, Seoul (KR); Bong-Hwan Cho, Seoul (KR)

Correspondence Address: BIRCH STEWART KOLASCH & BIRCH, PO BOX 747, FALLS CHURCH, VA, 22040-0747, US

(21) Appl. No.: 11/667,048

(22) PCT Filed: Apr. 21, 2006
(86) PCT No.: PCT/KR2006/001500

§ 371 Date: Oct. 31, 2007

(30) Foreign Application Priority Data
Apr. 25, 2005 (KR) 10 2005 0034057

(51) Int. Cl. A61K 31/44; C07D 211/60; A61P 31/04

(52) U.S. Cl. 514/352; 546/309

(57) ABSTRACT

The present invention relates to the novel antibacterial compounds having potent antibacterial activity as inhibitors of peptide deformylase. This invention further relates to pharmaceutically acceptable salts thereof, to processes for their preparation, and to pharmaceutical compositions containing them as an active ingredient.

### TECHNICAL FIELD

The present invention relates to the novel antibacterial compounds having potent antibacterial activity as inhibitors of peptide deformylase. This invention further relates to pharmaceutically acceptable salts thereof, to processes for their preparation, and to pharmaceutical compositions containing them as an active ingredient.

### BACKGROUND ART

Infectious diseases caused by bacteria, fungi and other parasitic organisms affect hundreds of millions of people worldwide and cause millions of deaths each year. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention are regarded primarily as effective against Gram-positive pathogens because of their particularly potent activity against such pathogens.

Recently published literature indicate that bacteria are rapidly acquiring resistance to well known antibiotics, including vancomycin and new agent such as linezolid (Staphylococcus aureus resistant to vancomycin—United States, 2002. MMWR (2002) 51(26): 565-567; linezolid resistance in a clinical isolate of Staphylococcus aureus. Lancet (2001) 358 (9277): 207-208).

Therefore, there is an urgent need to discover antibiotics with new modes of action.

Peptide deformylase (PDF), an essential enzyme involved in bacterial protein biosynthesis and maturation, is one of the few novel targets that is currently being pursued for antibacterial drug design. PDF is a unique metallopeptidase, which utilizes a ferrous ion (Fe²⁺) to catalyze the amide bond hydrolysis. In bacteria, protein synthesis starts with an N-formyl methionine (fMet), and as a result, all newly synthesized polypeptides carry a formylated N-terminus. PDF catalyzes the subsequent removal of the formyl group from the majority of those polypeptides, many of which undergo further N-terminal processing by methionine aminopeptidase (MAP) to produce mature proteins. Since protein synthesis in eukaryotic organisms dose not depend on N-formyl methionine (fMet) for initiation, PDF inhibitors are expected to act as a new class of antimicrobial and antibacterial agents. Numerous PDF inhibitors have been reported in recent years; essentially all of them are metal chelators. On the basis of the chelator structure, they can be classified into three different types: the thiols, the hydroxamic acids, and the N-formyl hydroxylamines.

Several PDF inhibitors have been reported in the literature some of which relevant are given here:

hydroxamic acid derivatives: WO 99/59568, WO 00/44373, WO 01/44178, WO 01/44179, WO 02/28829 and WO 02/081426

N-formyl hydroxylamines derivatives: WO 01/85160, WO 01/85170, WO 02/070540, WO 02/070541, WO 02/070653, WO 02/070654, WO 02/098901, WO 03/101442, WO 0035440, WO 99/39704, WO 00/35440, WO 00/58294, WO 00/61134, WO 01/10834, WO 01/10835, WO 03/089412 and WO 2004/033441

Although a wide variety of compounds described in prior art have been developed as inhibitors of peptide deformylase, they did not result in a clinically useful compound.

Though a variety of inhibitors have been prepared, there is a continuing need for potent peptide deformylase inhibitors useful in treating such diseases.

The present invention fulfills this need.

### DISCLOSURE OF INVENTION

### Technical Solution

The present invention relates to the novel hydroxamic acid derivatives having potent antibacterial activity as inhibitors of peptide deformylase. This invention further relates to processes for their preparation, to intermediates useful in their preparation, and to pharmaceutical compositions containing them as an active ingredient:

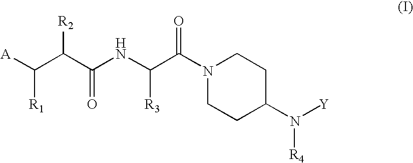

wherein, A is selected from the group of consisting of —C(═O)NHOH or —N(CHO)OH;

R₁ represents hydrogen, C₁₋₃ alkyl, C₄₋₆ cycloalkyl, halogen or hydroxy group;

R₂represents hydrogen, straight or branched C₁₋₆ alkyl, straight or branched C₁₋₆ alkenyl, C₄₋₆ cycloalkyl, C₄₋₆ heterocycle including nitrogen or oxygen, or benzyl group;

R₃ represents hydrogen, methyl, straight or branched C₁₋₆ alkyl, straight or branched C₁₋₆ alkenyl, C₄₋₆ cycloalkyl, phenyl or benzyl group;

R₄ represents hydrogen, straight or branched C₁₋₄ alkyl, C₁₋₄ alkenyl, hydroxy substituted C₄₋₆ cycloalkyl group; and

Y represents a group of formula (IIa), or (IIb), or (IIc):

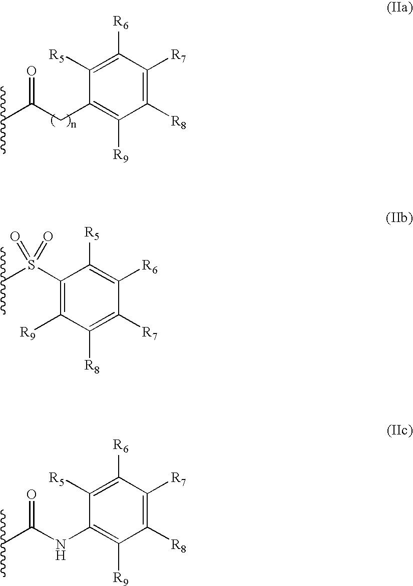

wherein, n is independently 0 or 1;

each of R₅, R₆, R₇, R₈ and R₉ is independently hydrogen, straight or branched C₁₋₃ alkyl, hydroxy, alkoxy, acyl, acyloxy, halogen (fluoro, chloro, bromo and iodo) cyano, nitro, amino, N,N-dimethylamino, phenyl, morpholinyl, or formyl group.

### BEST MODE FOR CARRYING OUT THE INVENTION

The present invention relates to the novel hydroxamic acid derivatives having potent antibacterial activity as inhibitors of peptide deformylase. This invention further relates to processes for their preparation, to intermediates useful in their preparation, and to pharmaceutical compositions containing them as an active ingredient:

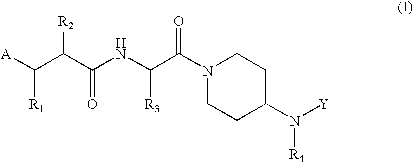

wherein, A is selected from the group of consisting of —C(═O)NHOH or —N(CHO)OH;

R₁ represents hydrogen, C₁₋₃ alkyl, C₄₋₆ cycloalkyl, halogen or hydroxy group;

R₂ represents hydrogen, straight or branched C₁₋₆ alkyl, straight or branched C₁₋₆ alkenyl, C₄₋₆ cycloalkyl, C₄₋₆ heterocycle including nitrogen or oxygen, or benzyl group;

R₃ represents hydrogen, methyl, straight or branched C₁₋₆ alkyl, straight or branched C₁₋₆ alkenyl, C₄₋₆ cycloalkyl, phenyl or benzyl group;

R₄ represents hydrogen, straight or branched C₁₋₄ alkyl, C₁₋₄ alkenyl, hydroxy substituted C₄₋₆ cycloalkyl group; and

Y represents a group of formula (IIa), or (IIb), or (IIc):

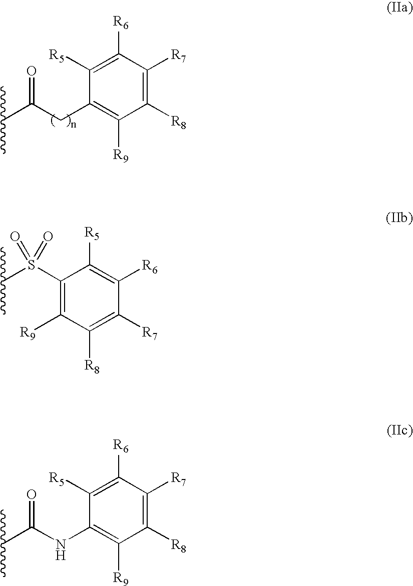

wherein, n is independently 0 or 1;

each of R₅, R₆, R₇, R₈ and R₉ is independently hydrogen, straight or branched C₁₋₃ alkyl, hydroxy, alkoxy, acyl, acyloxy, halogen (fluoro, chloro, bromo and iodo) cyano, nitro, amino, N,N-methylamino, phenyl, morpholinyl, or formyl group.

The compounds of this invention may possess one or more asymmetric centers because of the presence of asymmetric carbon atoms. Therefore, the invention includes all such racemic mixtures, optical isomers and diastereoisomers thereof.

A compounds of the invention may be administered in pharmaceutically acceptable salt forms, hydrate forms or solvate forms. Such salts include acid addition salts, formed with hydrochloric acid, hydrobromic acid, sulfuric acid, nitric acid, methanesulfonic acid, p-toluenesulfonic acid, phosphoric acid, acetic acid, pyruvic acid, citric acid, succinic acid, lactic acid, tartaric acid, fumaric acid, maleic acid, stearic acid and salicylic acid. Salts may also be formed with sodium, potassium, magnesium and calcium salts.

The present invention provides a process for preparing of formula (I), or pharmaceutically acceptable salt, hydrate or solvate thereof.

Compounds of the invention wherein A is —C(═O)NHOH group may be prepared by reacting a compound of formula (E) with hydroxylamine or an N- and/or O-protected hydroxylamine, and thereafter removing any N- or O-protecting groups:

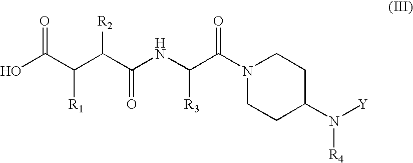

wherein, R₁, R₂, R₃, R₄ and Y are the same as defined above.

Reaction of formula (III) with hydroxylamine or an N- and/or O-protected hydroxylamine may be carried out according to the standard peptide coupling conditions.

The reaction is typically carried out in the presence of a coupling reagent (e.g. pentafluorophenol, N,O-methylhydroxylamine, DMAP/EDCI, EDCI/HOBt/NMM, etc.), in an appropriate solvent (e.g. tetrahydrofuran, dichloromethane, N,N-dimethylformamide, etc.).

Deprotection of benzyl group may be carried out in the presence of the hydrogenation catalyst, preferably a palladium catalyst (e.g. palladium on carbon or palladium black). The reaction can be achieved under a hydrogen atmosphere for about 2 to about 24 hours.

Deprotection of tert-butoxycarbonyl group may be carried out in the presence of an appropriate acid, such as hydrochloric acid or trifluoroacetic acid. The reaction can be achieved by stirring for about 2 to about 24 hours.

Compounds of formula (III) may be prepared by reacting a compound of formula (IV) with a compound of formula (Va) (or Vb, or Vc) or salt thereof.

Reaction of formula (IV) with a compound of formula (Va) (or Vb, or Vc) or salt thereof may be carried out according to the standard peptide coupling conditions.

The reaction is typically carried out in the presence of a coupling reagent (e.g. pentafluorophenol, N,O-methylhydroxylamine, DMAP/EDCI, EDCI/HOBt/NMM, etc.), in an appropriate solvent (e.g. tetrahydrofuran, dichloromethane, N,N-dimethylformamide, etc.):

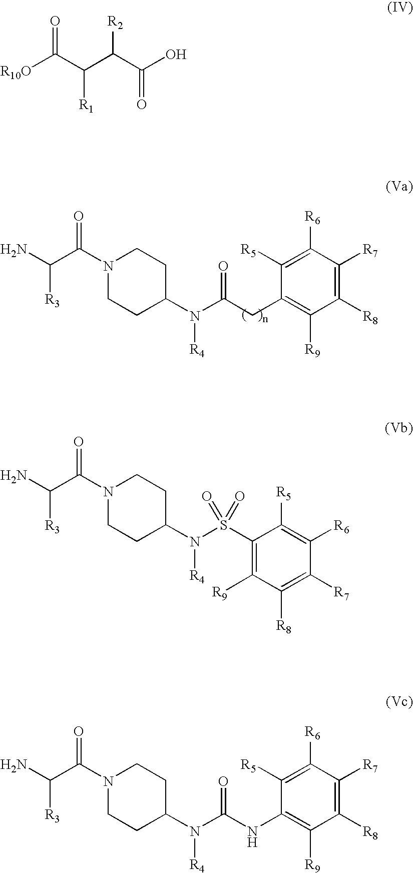

wherein, R₁, R₂, R₃, R₄, R₅, R₆, R₇, R₈, R₉ and n are the same as defined above and R is a hydroxy protecting group, such as methyl, ethyl, t-butyl and benzyl group.

Carboxylic acids of formula (IV) may be prepared according to any of a variety of methods described in the literature.

Also, compounds of the invention wherein A is —N(CHO)OH group may be prepared by reacting a compound of formula (VI) with a compound of formula (Va) (or Vb, or Vc) or salt thereof.

Reaction of formula (VI) with a compound of formula (Va) (or Vb, or Vc) or salt thereof may be carried out according to the standard peptide coupling conditions.

The reaction is typically carried out in the presence of a coupling reagent (e.g. pentafluorophenol, N,O-dimethylhydroxylamine, DMAP/EDCI, EDCI/HOBt/NMM, etc.), in an appropriate solvent (e.g. tetrahydrofuran, dichloromethane, N,N-dimethylformamide, etc.).

Deprotection of benzyl group may be carried out in the presence of the hydrogenation catalyst, preferably a palladium catalyst (e.g. palladium on carbon or palladium black). The reaction can be achieved under a hydrogen atmosphere for about 2 to about 24 hours.

Deprotection of tert-butoxycarbonyl group may be carried out in the presence of an appropriate acid, such as hydrochloric acid or trifluoroacetic acid. The reaction can be achieved by stirring for about 2 to about 24 hours:

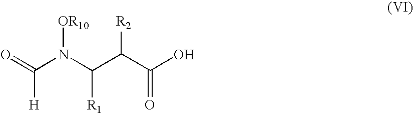

wherein, R₁, R₂ and R₁₀ are the same as defined above.

Carboxylic acids of formula (VI) may be prepared according to any of a variety of methods described in the literature.

The compound of formula (Va) (or Vb, or Vc) or salt thereof may be obtained by reacting a compound of formula (VII) with a compound of (VIIIa) (or VIIIb, or VIIIc) or salt thereof.

The reaction is typically carried out in the presence of a coupling reagent (e.g. pentafluorophenol, N,O-dimethylhydroxylamine, DMAP/EDCI, EDCI/HOBt/NMM, etc.), in an appropriate solvent (e.g. tetrahydrofuran, dichloromethane, N,N-dimethylformamide, etc.):

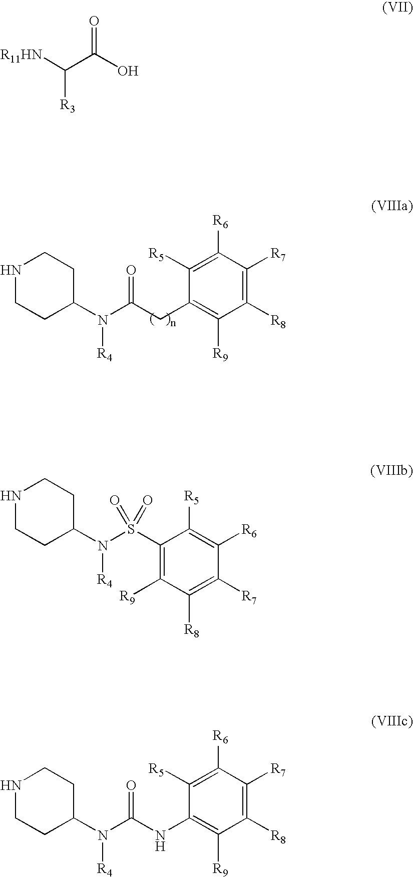

wherein, R₃, R₄, R₅, R₆, R₇, R₈, R₉ and n are the same as defined above and R₁₁ is a amino protecting group, such as tert-butoxycarbonyl, benzyloxycarbonyl or triphenylmethyl group.

The compound of formula (VIIIa) (or VIIIb, or VIIIc) or salt thereof may be obtained by reacting a compound of formula (IX) or salt thereof with a compound of formula (X) wherein Z is Cl(C═O) group. And also, reacting the compound of formula (IX) or salt thereof with a compound of formula (X) wherein Z is NH group may be carried out in the presence of a reagent such as triphosgen or 1,1′-carbonyldiimidazole.

Preferably, reacting the compound of formula (IX) or salt thereof with a compound of formula (X) may be carried out in an organic solvent such as dichloromethane, acetonitrile, tetrahydrofuran, dimethyl sulfoxide and toluene in the presence of a base such as N,N-diisopropylethylamine, triethylamine, N-ethylmorpholine:

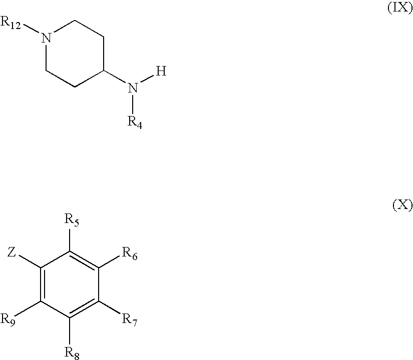

wherein, R₄, R₅, R₆, R₇, R₈ and R₉ are the same as defined above and R₁₂ is a amino protecting group, such as tert-butoxycarbonyl, benzyloxycarbonyl or triphenylmethyl group, and Z is selected from the group of consisting of Cl(C═O), Cl(C═O)CH₂, ClS(═O)₂, NH₂ or O═C═N.

The examples which follow illustrate embodiments of the invention but are not intended to limit the scope in any way.

General Procedure I

### Synthesis of N-[1-((S)-2-Amino-3,3 dimethyl-butyryl)-piperidin-4-yl]-4fluoro-benzamide hydrochloride (Scheme 1)

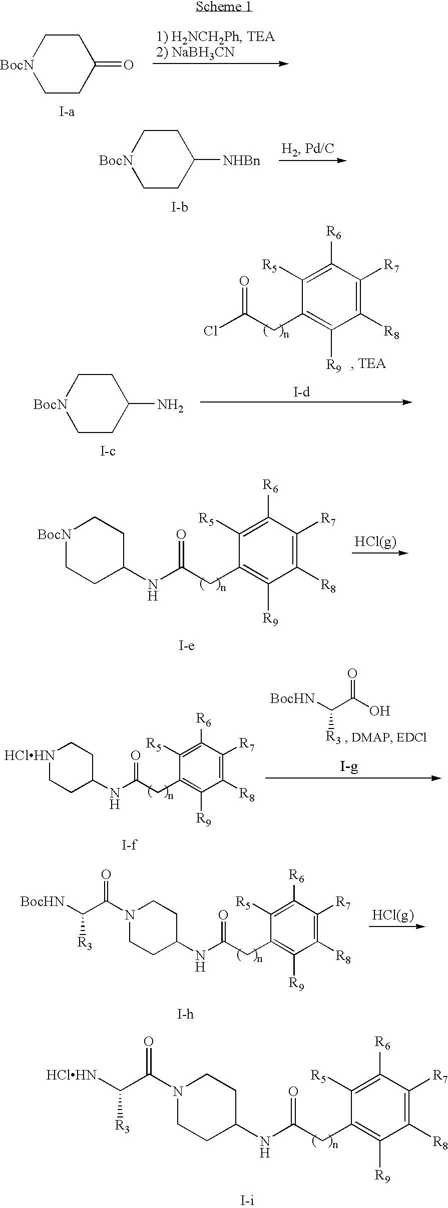

### Step 1: 4-Benzylamino-piperidine-1-carboxylic acid tert-butyl ester (I-b)

To a solution of tert-butyl-4-oxo-1-piperidinecarboxylate (25 g, 125.47 mmol) in anhydrous MeOH (600 mL) was added triethylamine (26.23 mL, 188.20 mmol) and benzylamine (20.56 mL, 188.20 mmol). The reaction mixture was heated to 65° C. for 5 h before adding sodium cyanoborohydride (15.77 g, 250.94 mmol) portionwise. The mixture was stirred for 48 h and filtered through Celite. The filtrate was evaporated to dryness and ethyl acetate was added. The organic phase was washed with saturated aqueous NaHCO₃, then H₂O, dried over MgSO₄ and evaporated to dryness under reduced pressure. The residue was purified by column chromatography to give the title compound as a pale yellow solid (30 g, 82%).

¹H-NMR(CDCl₃): δ 7.22-7.35 (m, 5H), 3.95-4.10 (m, 2H), 3.82(s, 2H), 2.75-2.83 (m, 2H), 2.61-2.71 (m, 1H), 1.84-1.88 (m, 2H), 1.45 (s, 9H), 1.24-1.39 (m, 2H).

### Step 2: 4-Amino-piperidine-1-carboxylic acid tert-butyl ester (I-c)

To a solution of compound I-b (25 g, 86.09 mmol) in ethanol (500 mL) was added 10% palladium on charcoal (8.62 g). The mixture was exposed to 7 atm of hydrogen until all of the starting material was consumed. The charcoal was removed by filtration and the filtrate was concentrated to give the title compound as a white crystalline solid (17 g, 98%).

¹H-NMR(CDCl₃): δ 3.90-4.10 (m, 2H), 2.66-2.79 (m, 3H), 1.69-1.73 (m, 2H), 1.38 (s, 9H), 1.10-1.21 (m, 2H).

### Step 3: 4-(4-Fluoro-benzoylamino)-piperidine-1-carboxylic acid tert-butyl ester (I-e)

A solution of compound I-c (5 g, 24.96 mmol) and triethylamine (4.7 mL, 33.70 mmol) in CH₂Cl₂ (50 mL) was cooled to 0° C. A solution of compound I-d (R₅═R₆═R₈═R₉═H, R₇═F, n=0, 5.1 g, 32.1 mmol) in CH₂Cl₂ (30 mL) was slowly added into the above reaction mixture. After stirring for 12 h at room temperature, the mixture was poured into H₂O and extracted with CH₂Cl₂. The organic layer was washed with aqueous saturated NaHCO₃ and brine and dried over MgSO₄. After removing the solvent in vacuo, the residue was purified by column chromatography to give the title compound as a white crystalline solid (7 g, 87%).

¹H-NMR(CDCl₃): δ 7.75-7.80(m, 2H), 7.07-7.27 (m, 2H), 4.05-4.19 (m, 3H), 2.85-2.93 (m, 2H), 1.97-2.04 (m, 2H), 1.46 (s, 9H), 1.39-1.44 (m, 2H).

### Step 4: 4-Fluoro-N-piperidin-4-yl-benzamide hydrochloride (I-f)

Compound I-e (5 g, 15.51 mmol) was dissolved in ethyl acetate (30 mL) and saturated with gaseous HCl, and the reaction mixture stirred until all of the starting material was consumed. The mixture was concentrated to give amine hydrochloride salt as a white crystalline solid which was used in next step without further purification (3.8 g, 95%).

¹H-NMR(D₂O): δ 7.57-7.64 (m, 2H), 7.01-7.11 (m, 2H), 3.93-4.03 (m, 1H), 3.34-3.41 (m, 2H), 2.92-3.07 (m, 2H), 2.02-2.11 (m, 2H), 1.61-1.75 (m, 2H).

### Step 5: {(S)-1-[4-(4Fluoro-benzoylamino)-piperidine-1-carbonyl]-2,2-dimethyl-propyl}-carbamic acid tertbutyl ester (I-h)

To a solution of I-g (R₃=tert-butyl, 3 g, 12.97 mmol) in CH₂Cl₂ (60 mL) at 0° C. was added successively compound I-f (3.7 g, 14.30 mmol), 4-dimethylaminopyridine (DMAP) (3.32 g, 27.17 mmol) and 1-ethyl-3-(3-dimethylaminopropyl)carbodiimide hydrochloride (EDCI) (2.74 g, 14.30 mmol). The mixture was stirred at room temperature overnight. The mixture was washed with aqueous saturated NaHCO₃ and brine and dried over MgSO₄. After removing the solvent in vacuo, the residue was purified by column chromatography to give the title compound as a white solid (5 g, 88%).

¹H-NMR(CDCl): δ 7.76-7.82 (m, 2H), 7.07-7.13 (m, 2H), 4.52-4.68 (m, 2H), 4.09-4.25 (m, 2H), 3.15-3.32 (m, 1H), 2.72-2.85 (m, 1H), 2.02-2.21 (m, 2H), 1.35-1.52 (m, 11H), 0.96-0.98 (d, 9H).

### Step 6: N-[1-((S)-2-Amino-3,3-dimethyl-butyryl)-piperidin-4-yl]-4-fluoro-benzamide hydrochloride (I-i)

Compound I-h (3 g, 6.936 mmol) was dissolved in ethyl acetate (40 mL) and saturated with gaseous HCl, and the reaction mixture stirred until all of the starting material was consumed. The mixture was concentrated to-give amine hydrochloride salt as a white crystalline solid which was used in next step without further purification (2.4 g, 93%).

¹H-NMR(D₂O): δ 7.57-7.62 (m, 2H), 7.02-7.09 (m, 2H), 4.20-4.35 (m, 1H), 3.93-4.01 (m, 3H), 3.16-3.30 (m, 1H), 2.77-2.95 (m, 1H), 1.82-1.95 (m, 2H), 1.35-1.53 (m, 2H), 0.93-0.96 (d, 9H).

General Procedure II

### Synthesis of 1-[1-((S)-2-Amino-3,3-dimethyl-butyryl)-piperidin-4yl]-3-(4-fluorophenyl)-urea hydrochloride (Scheme II)

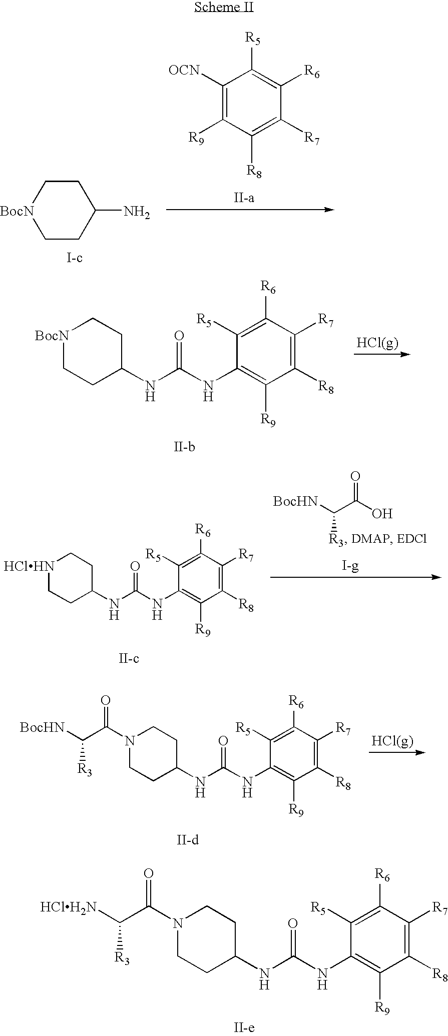

### Step 1: 4-[3-(4-Fluoro-phenyl)ureido]-piperidine-1-carboxylic acid tert-butyl ester (II-b)

To a solution of compound I-c (5 g, 24.96 mmol) in THF (60 mL) was added compound II-a (R₅═R₆═R₈═R₉═H, R₇═F, 4.35 g, 27.43 mmol). The reaction mixture was allowed to stir at room temperature for 1-2 hours. After removing the solvent in vacuo, the residue was purified by column chromatography to give the title compound as a white solid (6 g, 71%).

¹H-NMR(CDCl₃): δ 7.75-7.81(m, 2H), 7.08-7.27 (m, 2H), 4.09-4.22 (m, 3H), 2.85-2.95 (m, 2H), 1.98-2.04 (m, 2H), 1.47 (s, 9H), 1.41-1.45 (m, 2H).

### Step 2: 1-(4-Fluoro-phenyl)-3-piperidin-4-yl-urea hydrochloride (II-c)

Compound II-b (4 g, 11.85 mmol) was dissolved in ethyl acetate (50 mL) and saturated with gaseous HCl, and the reaction mixture stirred until all of the starting material was consumed. The mixture was concentrated to give amine hydrochloride salt as a white crystalline solid which was used in next step without further purification (3.1 g, 96%).

¹H-NMR(D₂O): δ 7.59-7.68 (m, 2H), 7.01-7.13 (m, 2H), 3.98-4.10 (m, 1H), 3.35-3.44 (m, 2H), 2.94-3.07 (m, 2H), 2.05-2.14 (m, 2H), 1.62-1.75 (m, 2H).

### Step 3: ((S)-1-{4-[3-(4-Fluoro-phenyl)ureido]-piperidine-1-carbonyl}-2,2-dimethylpropyl)-carbamic acid tert-butyl ester (II-d)

To a solution of I-g (R₃=tertbutyl, 2 g, 8.647 mmol) in CH₂Cl₂ (40 mL) at 0° C. was added successively compound II-c (2.6 g, 9.498 mmol), 4-dimethylaminopyridine (DMAP) (2.2 g, 18.007 mmol) and 1-ethyl-3-(3-dimethylaminopropyl)carbodiimide hydrochloride (EDCI) (1.8 g, 9.498 mmol). The mixture was stirred at room temperature overnight. The mixture was washed with aqueous saturated NaHCO₃ and brine and dried over MgSO₄. After removing the solvent in vacuo, the residue was purified by column chromatography to give the title compound as a white solid (3.5 g, 90%).

¹H-NMR(CDCl₃): δ 7.77-7.85 (m, 2H), 7.09-7.13 (m, 2H), 4.55-4.69 (m, 2H), 4.11-4.26 (m, 2H), 3.18-3.32 (m, 1H), 2.75-2.86 (m, 1H), 2.04-2.22 (m, 2H), 1.35-1.54 (m, 11H), 0.96-0.99 (d, 9H).

### Step 4: 1-[1-((S)-2-Amino-3,3-dimethyl-butyryl)-piperidin-4-yl]-3-(4-fluoro-phenyl)-urea hydrochloride (II-e)

Compound II-d (2.5 g, 5.549 mmol) was dissolved in ethyl acetate (35 mL) and saturated with gaseous HCl, and the reaction mixture stirred until all of the starting material was consumed. The mixture was concentrated to give amine hydrochloride salt as a white crystalline solid which was used in next step without further purification (2.1 g, 98%).

¹H-NMR(D₂O): δ 7.58-7.65 (m, 2H), 7.04-7.09 (m, 2H), 4.25-4.38 (m, 1H), 3.95-4.01 (m, 3H), 3.18-3.32 (m, 1H), 2.79-2.95 (m, 1H), 1.83-1.95 (m, 2H), 1.37-1.54 (m, 2H), 0.94-0.96 (d, 9H).

General Procedure III

### Synthesis of N-[1-((S)-2-Amino-3,3-dimethyl-butyryl)-piperidin-4-yl]-4-fluoro-benzenesulfonamide hydrochloride (Scheme III)

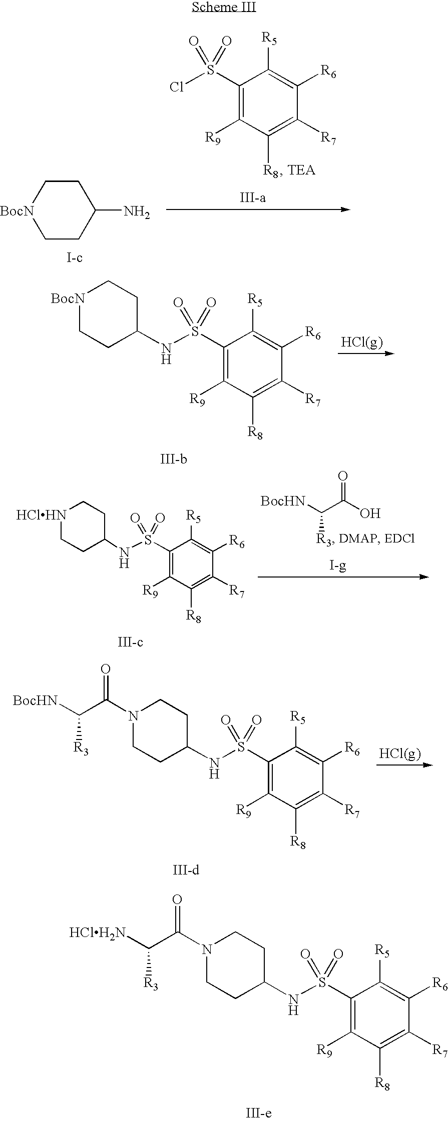

### Step 1: 4-(4-Fluoro-benzenesulfonylamino)-piperidine-1-carboxylic acid tert-butyl ester (III-b)

To a solution of compound I-c (10 g, 49.93 mmol) in acetone/distilled water (4/1, 250 mL) was added compound III-a (R₅═R₆═R₈═R₉═H, R₇═F; 9.72 g, 49.93 mmol). A solution of triethylamine (6.96 mL, 49.93 mmol) in distilled water was added and the reaction mixture allowed to stir at room temperature for 5 hours. The solvent was removed in vacuo and the residue dissolved in ethyl acetate. The organic layer was separated and the aqueous phase extracted with ethyl acetate. The combined organic extracts were dried over MgSO₄, filtered and the solvents removed in vacuo to give a crude product. The crude product was purified by column chromatography to give the title compound as a white solid (16 g, 89%).

¹H-NMR(CDCl₃): δ 7.75-7.81(m, 2H), 7.08-7.19 (m, 2H), 4.09-4.22 (m, 2H), 3.02-3.21 (m, 1H), 2.85-2.95 (m, 2R), 1.98-2.04 (m, 2H), 1.47 (s, 9H), 1.41-1.45 (m, 2H).

### Step 2: 4-Fluoro-N-piperidin-4-yl-benzenesulfonamide hydrochloride (III-c)

Compound III-b (10 g, 27.899 mmol) was dissolved in ethyl acetate (120 mL) and saturated with gaseous HCl, and the reaction mixture stirred until all of the starting material was consumed. The mixture was concentrated to give amine hydrochloride salt as a white crystalline solid which was used in next step without further purification (8 g, 97%).

¹H-NMR(D₂O): δ 7.61-7.68 (m, 2H), 7.10-7.17 (m, 2H), 3.34-3.44 (m, 2H), 3.01-3.15 (m, 1H), 2.93-3.09 (m, 2H), 2.04-2.15 (m, 2H), 1.63-1.75 (m, 2H).

### Step 3: {(S)-1-[4-(4-Fluoro-benzenesulfonylamino)-piperidine-1-carbonyl]-2,2-dimethyl-propyl}-carbamic acid tertbutyl ester (III-d)

To a solution of I-g (R₃=tert-butyl, 4.5 g, 19.456 mmol) in CH₂Cl₂ (90 mL) at 0° C. was added successively compound III-c (5.86 g, 21.407 mmol), 4-dimethylaminopyridine (DMAP) (4.99 g, 40.845 mmol) and 1-ethyl-3-(3-dimethylaminopropyl)carbodiimide hydrochloride (EDCI) (4.1 g, 21.407 mmol). The mixture was stirred at room temperature overnight. The mixture was washed with aqueous saturated NaHCO₃ and brine and dried over MgSO₄. After removing the solvent in vacuo, the residue was purified by column chromatography to give the title compound as a white solid (8 g, 87%).

¹H-NMR(CDCl₃): δ 7.81-7.86 (m, 2H), 7.09-7.14 (m, 2H), 4.56-4.66 (m, 2H), 4.10-4.27 (m, 2H), 3.01-3.12 (m, 1H), 2.74-2.88 (m, 1H), 2.04-2.19 (m, 2H), 1.36-1.54 (m, 11H), 0.95-0.97 (d, 9H).

### Step 4: N-[1-((S)-2-Amino-3,3-dimethyl-butyryl)-piperidin-4-yl]-4-fluoro-benzenesulfonamide hydrochloride (III-e)

Compound III-d (6 g, 12.723 mmol) was dissolved in ethyl acetate (80 mL) and saturated with gaseous HCl, and the reaction mixture stirred until all of the starting material was consumed. The mixture was concentrated to give amine hydrochloride salt as a white crystalline solid which was used in next step without further purification (5 g, 96%).

¹H-NMR(D₂O): δ 7.69-7.73 (m, 2H), 7.02-7.05 (m, 2H), 4.19-4.34 (m, 1H), 3.88-4.00 (m, 3H), 3.11-3.31 (m, 1H), 2.74-2.92 (m, 1H), 1.82-1.91 (m, 2H), 1.34-1.51 (m, 2H), 0.93-0.97 (d, 9H).

General Procedure IV

### Synthesis of (R)-2-Butyl-N¹-{(S)-1-[4-(4-fluoro-benzenesulfonylamino)-piperidine-1-carbonyl]-2,2-dimethyl-propyl)-N⁴-hydroxy-succinamide (Scheme IV)

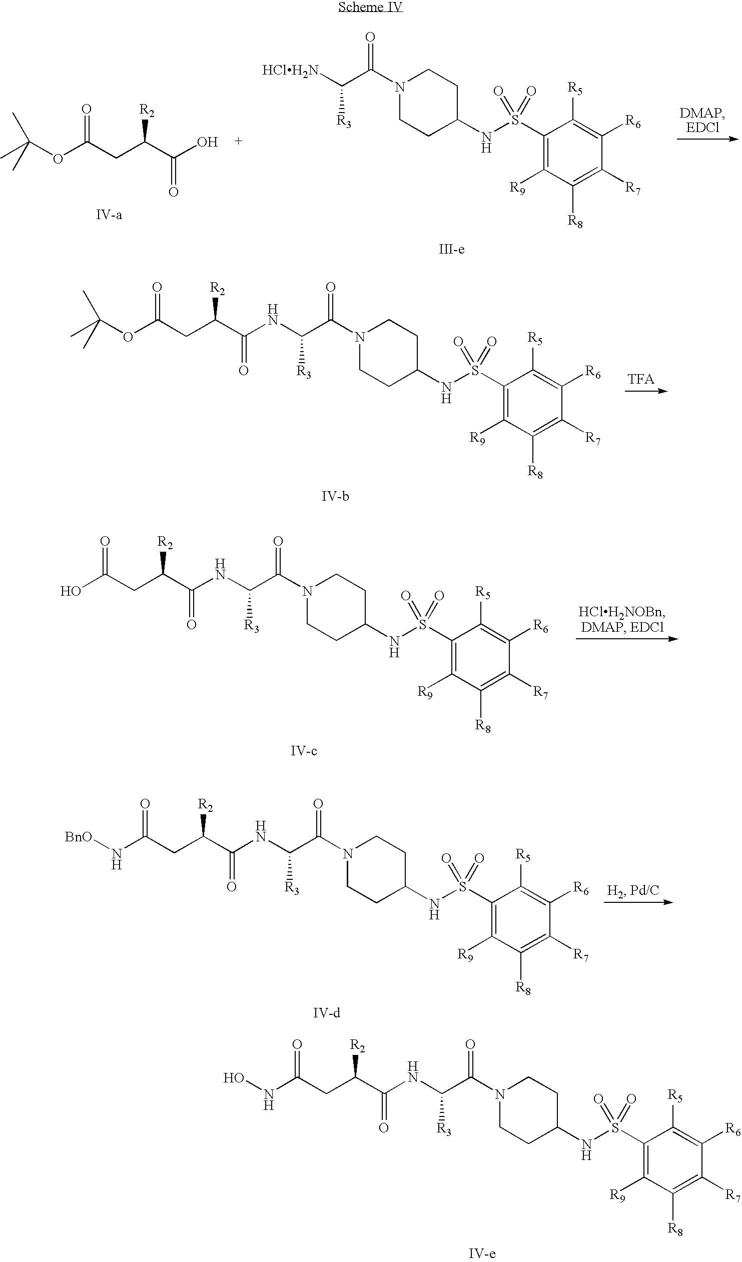

### Step 1: (R)-3-{(S)-1-[4-(4-Fluoro-benzenesulfonylamino)-piperidine-1-carbonyl]-2,2-dimethyl-propylcarbamoyl)-heptanoic acid tert-butyl ester (IV-b)

To a solution of IV-a (R₂=n-butyl, 0.5 g, 2.171 mmol) in CH₂Cl₂ (25 mL) at 0° C. was added successively compound III-e (R₃=tert-butyl, R₇═F, R₅═R₆═R₈═R₉═H, 1.06 g, 2.598 mmol), 4-dimethylaminopyridine (DMAP) (0.66 g, 5.402 mmol) and 1-ethyl-3-(3-dimethylaminopropyl)carbodiimide hydrochloride (EDCI) (0.5 g, 2.605 mmol). The mixture was stirred at room temperature overnight. The mixture was washed with 0.5N-HCl, aqueous saturated NaHCO₃ and brine, and dried over MgSO₄. After removing the solvent in vacuo, the residue was purified by column chromatography to give the title compound as a white solid (1 g, 79%).

¹H-NMR(CDCl₃): δ 7.72-7.79 (m, 2H), 7.07-7.14 (m, 2H), 4.90-4.94 (m, 1H), 4.51-4.70 (m, 1H), 4.19-4.25 (m, 2H), 3.19-3.31 (m, 1H), 2.72-2.81 (m, 1H), 2.53-2.63 (m, 2H), 2.30-2.38 (m, 1H), 2.09-2.23 (m, 2H), 1.25-1.71 (m, 19H), 0.99-1.00 (d, 9H), 0.83-0.89 (m, 3H).

### Step 2: (R)-3-((S)-1-[4-(4-Fluoro-benzenesulfonylamino)-piperidine-1-carbonyl]-2,2-dimethyl-propylcarbamoyl)-heptanoic acid (IV-c)

Compound IV-b (500 mg, 0.856 mmol) was dissolved in ethyl acetate (10 mL) and saturated with gaseous HCl, and the reaction mixture stirred until all of the starting material was consumed. The mixture was concentrated to give free acid as a white crystalline solid which was used in next step without further purification (430 mg, 96%).

¹H-NMR(CDCl₃): δ 7.87-7.93 (m, 2H), 7.23-7.30 (m, 2H), 4.76-4.83 (m, 1H), 4.27-4.40 (m, 1H), 3.91-4.18 (m, 2H), 3.12-3.22 (m, 1H), 2.69-2.81 (m, 2H), 2.46-2.49 (m, 1H), 2.38-2.43 (m, 1H), 2.18-2.25 (m, 1H), 1.75-1.81 (m, 2H), 1.12-1.52 (m, 8H), 0.90-0.94 (d, 9H), 0.77-0.81 (m, 3H).

### Step 3: (R)-N⁴-Benzyloxy-2-butyl-N¹-{(S)-1-[4-(4-fluoro-benzenesulfonylamino)-piperidine-1-carbonyl]-2,2-dimethyl-propyl}-succinamide (IV-d)

To a solution of IV-c (100 mg, 0.189 mmol) in CH₂Cl₂ (10 mL) at 0° C. was added successively O-benzylhydroxylamine hydrochloride (36.3 mg, 0.227 mmol), 4-dimethylaminopyridine (DMAP) (57.9 mg, 0.473 mmol) and 1-ethyl-3-(3-dimethylaminopropyl)carbodiimide hydrochloride (EDCI) (43.6 mg, 0.227 mmol). The mixture was stirred at room temperature overnight. The mixture was washed with 0.5N-HCl, aqueous saturated NaHCO₃ and brine, and dried over MgSO₄. After removing the solvent in vacuo, the residue was purified by column chromatography to give the title compound as a white solid (100 mg, 84%).

¹H-NMR(CDCl₃): δ 7.74-7.78 (m, 2H), 7.26-7.37 (m, 5H), 7.06-7.14 (m, 2H), 4.81-4.89 (m, 3H), 4.51-4.70 (m, 1H), 4.20-4.25 (m, 2H), 3.11-3.35 (m, 1H), 2.72-2.80 (m, 2H), 1.90-1.31 (m, 4H), 1.25-1.53 (m, 8H), 0.99-1.00 (d, 9H), 0.85-0.89 (m, 3H).

### Step 4: (R)-2-Butyl-N¹-{(S)-1-[4-(4-fluoro-benzenesulfonylamino)-piperidine-1-carbonyl]-2,2-dimethyl-propyl)-N⁴-hydroxy-succinamide (IV-e)

To a solution of compound IV-d (50 mg, 0.079 mmol) in ethanol (5 mL) was added 10% palladium on charcoal (8.5 mg). A balloon of hydrogen was placed over the reaction mixture, and it was stirred for 8 hours. The charcoal was removed by filtration and the filtrate was concentrated to give a crude product. The crude product was purified by column chromatography to give the title compound as a pale yellow solid (30 mg, 70%).

¹H-NMR(DMSO-d₆): δ 7.91 (m, 2H), 7.26 (dt, 2H), 4.78 (t, 1H), 4.37 (m, 1H), 4.10 (m, 2H), 3.15 (t, 1H), 2.74 (m, 2H), 2.02-2.14 (m, 2H), 1.83 (br s, 2H), 1.16-1.38 (m, 8H), 0.77-0.95 (m, 12H).

General Procedure V

### Synthesis of N-(1-{(S)-2-[(R)-2-Cyclopentylmethyl-3-(formyl-hydroxy-amino)-propionylamino]-3,3-dimethyl-butyryl}-piperidin-4-yl)-4-fluoro-benzamide (Scheme V)

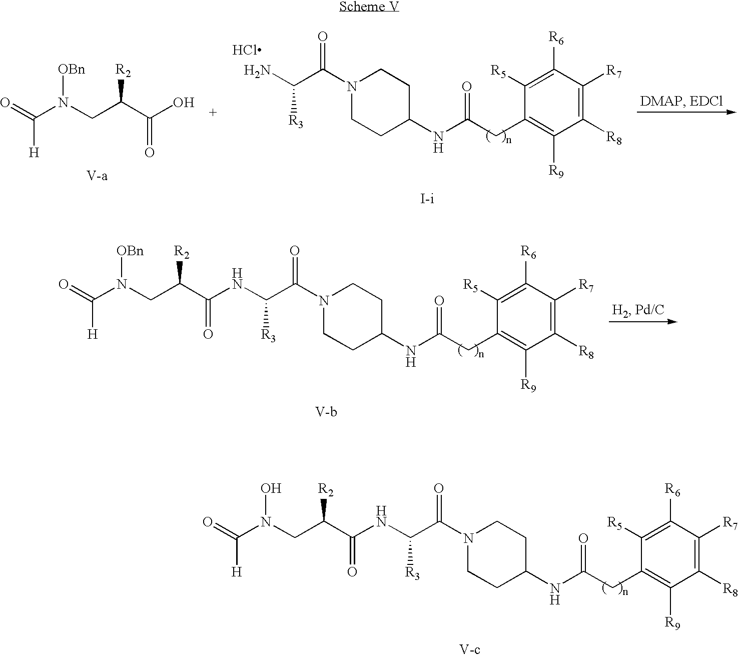

### Step 1: N-(1-{(S)-2-[(R)-3-(Benzyloxy-formyl-amino)-2-cyclopentylmethyl-propionylamino]-3,3dimethyl-butyryl)-piperidin-4-yl)-4-fluoro-benzamide (V-b)

To a solution of V-a (R₂=cyclopentylmethyl, 638 mg, 2.090 mmol) in CH₂Cl₂ (25 mL) at 0° C. was added successively compound I-i (R₃=tert-butyl, R₅═R₆═R₈═R₉═H, R₇═F, n=0, 855 mg, 2.299 mmol), 4-dimethylaminopyridine (DMAP) (562 mg, 4.599 mmol) and 1-ethyl-3-(3dimethylaminopropyl)carbodiimide hydrochloride (EDCI) (440 mg, 2.299 mmol). The mixture was stirred at room temperature overnight. The mixture was washed with 0.5N-HCl, aqueous saturated NaHCO₃ and brine, and dried over MgSO₄. After removing the solvent in vacuo, the residue was purified by column chromatography to give the title compound as a white solid (950 mg, 73%).

¹H-NMR(CDCl₃): δ 8.11 (s, 0.3H), 7.75-7.79 (m, 2.7H), 7.37 (m, 5H), 7.06-7.13 (m, 2H), 4.78-4.90 (m, 1H), 4.49-4.65 (m, 1H), 4.00-4.15 (m, 3H), 3.56-3.82 (m, 1H), 3.14-3.23 (m, 1H), 2.67-2.85 (m, 2H), 1.23-2.18 (m, 13H), 0.92-1.11 (m, 11H).

### Step 2: N-(1-{(S)-2-[(R)-2-Cyclopentylmethyl-3-(formyl-hydroxy-amino)-propionylamino]-3,3-dimethyl-butyryl}-piperidin-4-yl)-4-fluoro-benzamide (V-c)

To a solution of compound Vb (100 mg, 0.160 mmol) in ethanol (5 mL) was added 10% palladium on charcoal (17.2 mg). A balloon of hydrogen was placed over the reaction mixture, and it was stirred for 8 hours. The charcoal was removed by filtration and the filtrate was concentrated to give a crude product. The crude product was purified by column chromatography to give the title compound as a pale yellow solid (55 mg, 64%).

¹H-NMR(CDCl₃): δ 8.33 (s, 0.3H), 7.79-7.82 (m, 2.7H), 7.07-7.12 (m, 2H), 4.82-4.96 (m, 1H), 4.61-4.65 (m, 1H), 4.00-4.15 (m, 3H), 3.57-3.81 (m, 1H), 3.16-3.25 (m, 1H), 2.64-2.87 (m, 2H), 1.24-2.18 (m, 13H), 0.97-1.11 (m, 11H).

### Mode for the Invention

### EXAMPLE 1

### (R)-N¹-[(S)-1-(4-Benzoylamino-piperidine-1-carbonyl)-2,2-dimethyl-propyl]-2-butyl-N⁴-hydroxy-succinamide

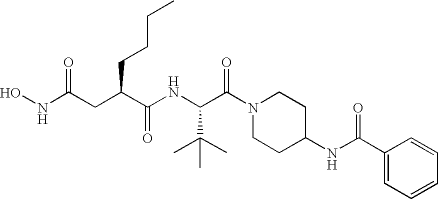

The title compound was prepared from (R)-2-butyl-succinic acid 4-tertbutyl ester IV-a (R₂=n-butyl) and N-[1-((S)-2-amino-3,3dimethyl-butyryl)-piperidin-4-yl]-benzamide hydrochloride I-i (R₃=tert-butyl, n=0, R₅═R₆═R₇═R₈═R₉═H) according to General procedure IV.

¹H-NMR(DMSO-d₆): δ 7.73-7.83 (m, 2H), 7.43-7.51 (m, 3H), 4.78 (t, 1H), 4.37 (t, 1H), 4.00-4.15 (m, 2H), 3.15 (t, 1H), 2.75 (m, 2H), 1.98-2.20 (m, 2H), 1.82 (br s, 2H), 1.14-1.55 (m, 8H), 0.77-0.95.

### EXAMPLE 2

### (R)-N¹-{(S)-1-[(4-Bromo-benzoylamino)-piperidine-1-carbonyl]-2,2-dimethyl-propyl]-2-butyl-N⁴-hydroxy-succinamide

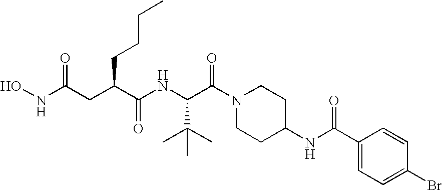

The title compound was prepared from (R)-2butyl-succinic acid 4-tertbutyl ester IV-a (R₂=n-butyl) and N-[1-((S)-2-amino-3,3-dimethyl-butyryl)-piperidin-4-yl]-4-bromo-benzamide hydrochloride I-i (R₃=tert-butyl, n=0, R₅═R₆═R₈═R₉═H, R₇═Br) according to General procedure IV.

¹H-NMR(DMSO-d₆): δ 7.73-7.83 (m, 2H), 7.44-7.50 (m, 2H), 4.78 (t, 1H), 4.37 (t, 1H), 4.03-4.11 (m, 2H), 3.14 (t, 1H), 2.75 (m, 2H), 1.98-2.20 (m, 2H), 1.82 (br s, 2H), 1.16-1.34 (m, 8H), 0.77-0.95 (m, 12H).

### EXAMPLE 3

### (R)-2-Butyl-N³-{(S)-2,2-dimethyl-1-[4-(4-trifluoromethyl-benzoylamino)-piperidine-1-carbonyl]-propyl}-N⁴-hydroxy-succinamide

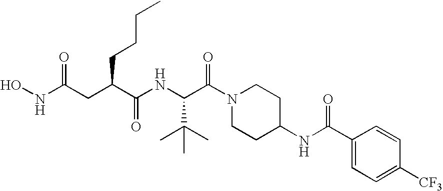

The title compound was prepared from (R)-2-butyl-succinic acid 4-tert-butyl ester IV-a (R₂=n-butyl) and N-[1-((S)-2-amino-3,3-dimethyl-butyryl)-piperidin-4-yl]-4-trifluoromethyl-benzamide hydrochloride I-i (R₃=tert-butyl, n=0, R₅═R₆═R₈═R₉═H, R₇═CF₃) according to General procedure IV.

¹H-NMR(DMSO-d₆): δ 8.03 (d, 2H), 7.82 (d, 2H), 4.78 (t, 1H), 4.31-4.42 (m, 1H), 4.03-4.11 (m, 2H), 3.13 (t, 1H), 2.71-2.74 (m, 2H), 2.14-2.20 (m, 1H), 1.97-2.04 (m, 1H), 1.85 (br s, 2H), 1.16-1.35 (m, 8H), 0.76-0.95 (m, 12H).

### EXAMPLE 4

### (R)-2-Butyl-N¹-[(S)-2,2-dimethyl-1-(4-phenylacetylamino-piperidine-1-carbonyl)-propyl]-N⁴-hydroxy-succinamide

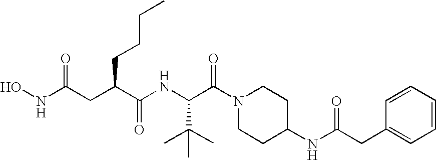

The title compound was prepared from (R)-2-butyl-succinic acid 4-tert-butyl ester IV-a (R₂=n-butyl) and N-[1-((S)-2-amino-3,3-dimethyl-butyryl)-piperidin-4-yl]-2-phenyl-acetamide hydrochloride I-i (R₃=tert-butyl, n=1, R₅═R₆═R₇═R₈═R₉═H) according to General procedure IV.

¹H-NMR(DMSO-d₆): δ 7.22-7.27 (m, 5H), 4.76 (m, 1H), 4.03 (m, 3H), 3.35 (d, 2H), 3.16 (m, 1H), 2.77 (m, 2H), 1.98-2.12 (m, 2H), 1.72 (br s, 2H), 1.16 (m, 8H), 0.76-0.93 (m, 12H).

### EXAMPLE 5

### (R)-N¹-[(S)-1-(4-Benzoylamino-piperidine-1-carbonyl)-2,2-dimethyl-propyl]- N⁴-hydroxy-2-isobutyl-succinamide

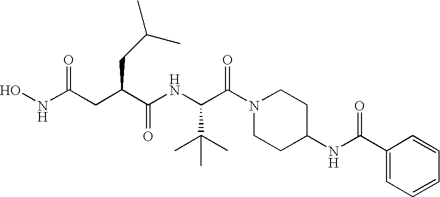

The title compound was prepared from (R)-2-isobutyl-succinic acid 4-tert-butyl ester IV-a (R₂=isobutyl) and N-[1-((S)-2-amino-3,3-dimethyl-butyryl)-piperidin4-yl]-benzamide hydrochloride I-i (R₃=tert-butyl, n=0, R₅═R₆═R₇═R₈═R₉═H) according to General procedure IV.

¹H-NMR(DMSO d₆): δ 7.69-7.80 (m, 2H), 7.34-7.46 (m, 3H), 4.71 (t, 1H), 4.23-4.35 (m, 1H), 3.91-4.02 (m, 2H), 3.07 (t, 1H), 2.58-2.77 (m, 2H), 1.89-2.15 (m, 2H), 1.74 (m, 2H), 1.28-1.48 (m, 4H), 1.00-1.03 (m, 1H), 0.70-0.88 (m, 15H).

### EXAMPLE 6

### (R)-N¹-{(S)-1-[4-(4-Bromo-benzoylamino)-piperidine-1-carbonyl]-2,2-dimethyl-propyl)-N⁴-hydroxy-2-isobutyl-succinamide

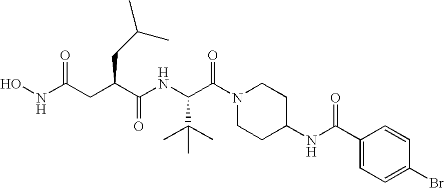

The title compound was prepared from (R)-2-isobutyl-succinic acid 4-tert-butyl ester IV-a (R=iso-butyl) and N-[1-((S)-2-amino-3,3-dimethyl-butyryl)-piperidin-4-yl]-4-bromo-benzamide hydrochloride I-i (R₃=tert-butyl, n=0, R₅═R₆═R₈═R₉═H, R₇═Br) according to General procedure IV.

¹H-NMR(DMSO-d₆): δ 7.68-7.79 (m, 2H), 7.34-7.46 (m, 2H), 4.71 (t, 1H), 4.22-4.35 (m, 1H), 3.91-4.02 (m, 2H), 3.07 (m, 11H), 2.59-2.77 (m, 2H), 1.89-2.09 (m, 2H), 1.74-1.83 (m, 2H), 1.15-1.65 (m, 4H), 1.00-1.04 (m, 1H), 0.70-0.88 (m, 15H).

### EXAMPLE 7

### (R)-N¹-[(S)-2,2-Dimethyl-1-(4-phenylacetylamino-piperidine-1-carbonyl)-propyl]-N⁴-hydroxy-2-isobutyl-succinamide

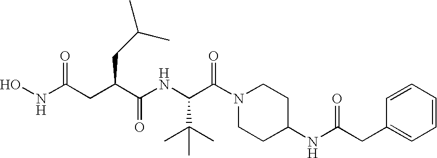

The title compound was prepared from (R)-2isobutyl-succinic acid 4-tert-butyl ester IV-a (R₂=isobutyl) and N-[1-((S)-2-amino-3,3-dimethyl-butyryl)-piperidin-4-yl]-2-phenyl-acetamide hydrochloride I-i (R₃=tert-butyl, n=1, R₅═R₆═R₇═R₈═R₉═H) according to General procedure IV.

¹H-NMR(DMSO-d₆): δ 7.14-7.20 (m, 5H), 4.67 (t, 1H), 3.93-4.18 (m, 2H), 3.67 (br s, 1H), 3.28 (d, 2H), 3.04 (m, 1H), 2.62-2.74 (m, 2H), 1.91-2.02 (m, 2H), 1.67 (m, 2H), 1.03-1.33 (m, 5H), 0.68-0.86 (m, 15H).

### EXAMPLE 8

### (R)-N¹-[(S)-1-(4-Benzoylamino-piperidine-1-carbonyl)-2,2-dimethyl-propyl]-2-cyclopentylmethyl-N⁴-hydroxy-succinamide

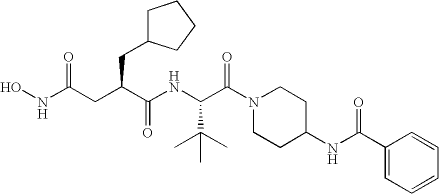

The tide compound was prepared from (R)-2-cyclopentylmethyl-succinic acid 4-tert-butyl ester IV-a (R₂=cyclopentylmethyl) and N-[1-((S)-2-amino-3,3-dimethyl-butyryl)-piperidin-4-yl]benzamide hydrochloride I-i (R₃=tert-butyl, n=0, R₅═R₆═R₇═R₈═R₉═H) according to General procedure IV.

¹H-NMR(CDCl₃): δ 7.78 (d, 2H), 7.41-7.52 (m, 3H), 4.83-4.90 (m, 1H), 4.56 (dd, 1H), 4.11-4.23 (m, 2H), 3.18-3.30 (m, 1H), 2.70-2.89 (m, 2H), 2.31 (d, 2H), 2.05 (m, 2H), 1.39-1.72 (m, 11H), 1.00 (m, 11H).

### EXAMPLE 9

### (R)-N¹-{(S)-1-[4-(4-Bromo-benzoylamino)-piperidine-1-carbonyl]-2,2-dimethyl-propyl}-2-cyclopentylmethyl-N⁴-hydroxy-succinamide

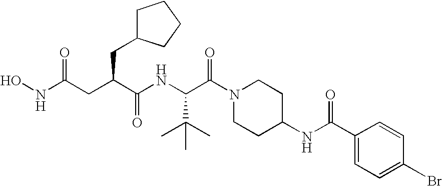

The title compound was prepared from (R)-2-cyclopentylmethyl-succinic acid 4-tert-butyl ester IV-a (R₂=cyclopentylmethyl) and N-[1-((S)-2-amino-3,3-dimethyl-butyryl)-piperidin-4-yl]-4bromo-benzamide hydrochloride I-i (R₃=tert-butyl, n=0, R₅═R₆═R₈═R₉═H, R₇═Br) according to General procedure IV.

¹H-NMR(CDCl₃): δ 7.78 (d, 2H), 7.40-7.52 (m, 2H), 4.83-4.90 (m, 1H), 4.56 (dd, 1H), 4.11-4.22 (m, 2H), 3.17-3.30 (m, 1H), 2.70-2.83 (m, 2H), 2.31-2.42 (m, 2H), 2.05 (m, 2H), 1.51-1.72 (m, 11H), 0.99 (m, 11H).

### EXAMPLE 10

### (R)-2-Cyclopentylmethyl-N¹-[(S)-2,2-dimethyl-1-(4-phenylacetylamino-piperidine-1-carbonyl)-propyl]-N⁴-hydroxy-succinamide

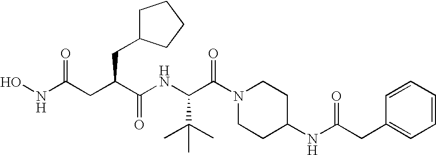

The title compound was prepared from (R)-2-cyclopentylmethyl-succinic acid 4-tert-butyl ester IV-a (R₂=cyclopentylmethyl) and N-[1-((S)-2-amino-3,3-dimethyl-butyryl)-piperidin-4-yl)-2-phenyl-acetamide hydrochloride I-i (R₃=tert-butyl, n=1, R₅═R₆═R₇═R₈═R₉═H) according to General procedure IV.

¹H-NMR(DMSO-d₆): δ 7.24-7.27 (m, 5H), 4.79 (t, 1H), 4.03-4.30 (m, 2H), 3.73 (m, 1H), 3.36 (d, 2H), 3.13-3.17 (m, 1H), 2.69-2.80 (m, 2H), 2.02-2.10 (m, 2H), 1.27-1.74 (m, 13H), 0.90-0.94 (m, 11H).

### EXAMPLE 11

### (R)-N¹-((S)-1-[4(4-Cyano-benzoylamino)-piperidine-1-carbonyl]-2,2-dimethyl-propyl}-N⁴-hydroxy-2-isobutyl-succinamide

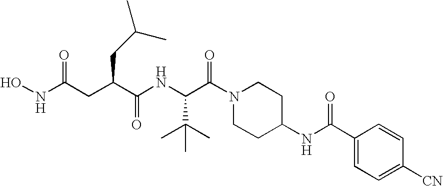

The title compound was prepared from (R)-2-isobutyl1-succinic acid 4-tert-butyl ester IV-a (R₂=iso-butyl) and N-[1-((S)-2-amino-3,3-dimethyl-butyryl)-piperidin4-yl]-4-cyano-benzamide hydrochloride I-i (R₃=tert-butyl, n=0, R₅═R₆═R₈═R₉═H, R₇═CN) according to General procedure IV.

¹H-NMR(DMSO-d₆): δ 7.75 (d, 2H), 7.25 (d, 2H), 4.79 (t, 1H), 4.25-4.50 (m, 1H), 3.90-4.20 (m, 2H), 3.16-3.18 (m, 1H), 2.86 (m, 2H), 1.99-2.13 (m, 2H), 1.81 (m, 2H), 1.42 (m, 4H), 1.10 (m, 1H), 0.79-0.96 (m, 15H).

### EXAMPLE 12

### (R)-N¹-{(S)-2,2-Dimethyl-1-[4-(4-trifluoromethyl-benzoylamino)-piperidine-1-carbonyl]-propyl}-N⁴-hydroxy-2-isobutyl-succinamide

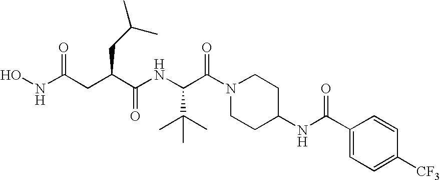

The title compound was prepared from (R)-2-isobutyl-succinic acid 4-tert-butyl ester IV-a (R₂=isobutyl) and N-[1-((S)-2-amino-3,3-dimethyl-butyryl)-piperidin-4-yl]-4-trifluoromethyl-benzamide hydrochloride I-i (R₃=tert-butyl, n=0, R₅═R₆═R₈═R₉═H. R₇═CF₃) according to General procedure IV.

¹H-NMR(DMSO-d₆): δ 8.04 (d, 2H), 7.84 (d, 2H), 4.80 (t, 1H), 4.25-4.50 (m, 1H), 3.90-4.20 (m, 2H), 3.16 (m, 1H), 2.72-2.86 (m, 2H), 1.99-2.14 (m, 2H), 1.87 (br s, 2H), 1.40 (m, 4H), 1.10 (m, 1H), 0.79-0.96 (m, 15H).

### EXAMPLE 13

### (R)-2-Cyclopentylmethyl-N¹-{(S)-2,2-dimethyl-1-[4-(4-trifluoromethyl-benzoylamino)-piperidine-1-carbonyl]-propyl}-N⁴-hydroxy-succinamide

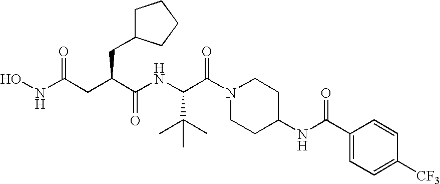

The title compound was prepared from (R)-2-cyclopentylmethyl-succinic acid 4-tert-butyl ester Iv-a (R₂=cyclopentylmethyl) and N-[1-((S)-2-amino-3,3-dimethyl-butyryl)-piperidin-4-yl]-4-trifluoromethyl-benzamide hydrochloride I-i (R₃=tert-butyl, n=0, R₅═R₆═R₈═R₉═H, R₇═CF₃) according to General procedure IV.

¹H-NMR(CDCl₃): δ 7.82-7.91 (m, 2H), 7.66 (d, 2H), 4.85-4.94 (m, 1H), 4.50-4.67 (m, 1H), 4.11-4.23 (m, 2H), 3.16-3.29 (m, 1H), 2.64-2.89 (m, 2H), 2.34-2.43 (m, 2H), 2.05 (m, 3H), 1.26-1.67 (m, 10H), 0.97 (m, 11H).

### EXAMPLE 14

### (R)-N¹-1-{(S)-1-[4-(4-Cyano-benzoylamino)-piperidine-1-carbonyl]-2,2-dimethyl-propyl}-2-cyclopentylmethyl-N⁴-hydroxy-succinamide

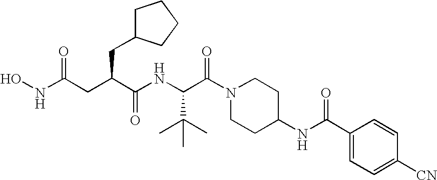

The title compound was prepared from (R)-2-cyclopentylmethyl-succinic acid 4-tert-butyl ester IV-a (R₂=cyclopentylmethyl) and N-[1-((S)-2-amino-3,3-dimethyl-butyryl)-piperidin-4-yl]-4-cyano-benzamide hydrochloride I-i (R₃=tertbutyl, n=0, R₅═R₆═R₈═R₉═H, R₇═CN) according to General procedure IV.

¹H-NMR(CDCl₃): δ 7.61-7.70 (m, 2H), 7.20 (d, 2H), 4.82-4.94 (m, 1H), 4.57 (dd, 1H), 4.11-4.22 (m, 2H), 3.15-3.33 (m, 1H), 2.64-2.88 (m, 2H), 2.38 (m, 2H), 1.28-2.18 (m, 13H), 0.97 (m, 1H).

### EXAMPLE 15

### (R)-2-Cyclopentylmethyl-N¹-{(S)-1-[4-(4-fluoro-benzoylamino)-piperidine-1-carbonyl]-2,2-dimethyl-propyl}-N⁴-hydroxy-succinamide

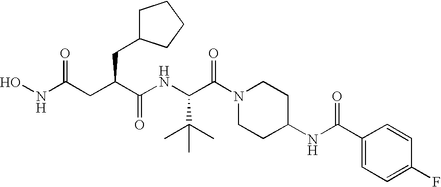

The title compound was prepared from (R)-2-cyclopentylmethyl-succinic acid 4-tert-butyl ester IV-a (R₂=cyclopentylmethyl) and N-[1-((S)-2-amino-3,3-dimethyl-butyryl)-piperidin-4-yl]-4-fluoro-benzamide hydrochloride I-i (R₃=tert-butyl, n=0, R₅═R₆═R₈═R₉═H, R₇═F) according to General procedure IV.

¹H-NMR(CDCl₃): δ 7.78 (m, 2H), 7.04-7.10 (m, 2H), 4.86-4.89 (m, 1H), 4.48-4.66 (m, 1H), 4.11-4.18 (m, 2H), 3.18-3.23 (m, 1H), 2.64-2.92 (m, 2H), 2.05-2.44 (m, 4H), 1.26-1.68 (m, 11H), 0.96 (m, 11H).

### EXAMPLE 16

### (R)-2-Butyl-N¹-{(S)-1 -[4-(4-fluoro-benzoylamino)-piperidine-1-carbonyl]-2,2-dimethyl-propyl}-N⁴-hydroxy-succinamide

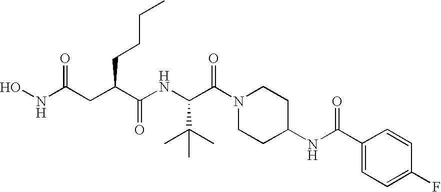

The title compound was prepared from (R)-2-butyl-succinic acid 4-tert-butyl ester IV-a (R₂=n-butyl) and N-[1-((S)-2-amino-3,3-dimethyl-butyryl)-piperidin4-yl]-4-fluoro-benzamide hydrochloride I-i (R₃=tert-butyl, n=0, R₅═R₆═R₈═R₉═H, R₇═F) according to General procedure IV.

¹H-NMR(DMSO-d₆): δ 7.91 (m, 2H), 7.26 (dt, 2H), 4.78 (t, 1H), 4.37 (m, 1H), 4.10 (m, 2H), 3.15 (t, 1H), 2.74 (m, 2H), 2.02-2.14 (m, 2H), 1.83 (br s, 2H), 1.16-1.38 (m, 8H), 0.77-0.95 (m, 12H).

### EXAMPLE 17

### (R)-N¹-{(S)-1-[4-(4-Fluoro-benzoylamino)-piperidine-1-carbonyl]-2,2-dimethyl-propyl}-N⁴-hydroxy-2-isobutyl-succinamide

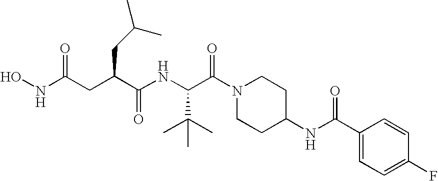

The title compound was prepared from (R)-2-isobutyl-succinic acid 4-tertbutyl ester IV-a (R₂=iso-butyl) and N-[1-((S)-2-amino-3,3-dimethyl-butyryl)-piperidin-4-yl]-4-fluoro-benzamide hydrochloride I-i (R₃=tert-butyl, n=0, R₅═R₆═R₈═R₉═H, R₇═F) according to General procedure IV.

¹H-NMR(DMSO-d₆): δ 7.91 (m, 2H), 7.26 (t, 2H), 4.78 (t, 1H), 4.37 (m, 1H), 4.11 (m, 2H), 3.14 (t, 1H), 2.70-2.84 (m, 2H), 1.97-2.13 (m, 2H), 1.83 (br s, 2H), 1.39 (m, 4H), 1.09 (m, 1H), 0.77-0.95 (m, 15H).

### EXAMPLE 18

### (R)-2-Butyl-N¹-{(S)-2,2-dimethyl-1-[4-(4-methyl-benzoylamino)-piperidine-1-carbonyl]-propyl}-N⁴-hydroxy-succinamide

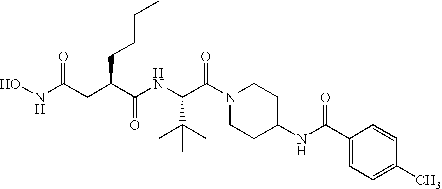

The title compound was prepared from (R)-2-butyl-succinic acid 4-tert-butyl ester IV-a (R₂=n-butyl) and N-[1-((S)-2-amino-3,3dimethyl-butyryl)-piperidinyl]-4-yl]-4-methyl-benzamide hydrochloride I-i (R₃=tert-butyl, n=0, R₅═R₆═R₈═R₉═H, R₇═CH₃) according to General procedure IV.

¹H-NMR(CD₃OD): δ 7.72 (t, 2H), 7.25 (d, 2H), 4.89-4.94 (m, 1H), 4.54 (dd, 1H), 4.08-4.26 (m, 2H), 3.21-3.31 (m, 1H), 2.74-2.86 (m, 2H), 2.37 (s, 3H), 2.17-2.41 (m, 2H), 2.01 (t, 2H), 1.21-1.64 (m, 8H), 0.86-1.04 (m, 12H).

### EXAMPLE 19

### (R)-2-Butyl-N⁴-hydroxy-N¹-{(S)-1-[4-(4-methoxy-benzoylamino)-piperidine-1-carbonyl]-2,2-dimethyl-propyl}-succinamide

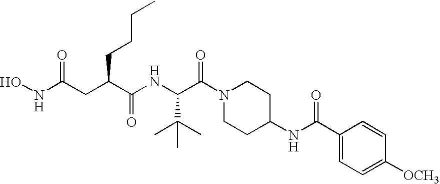

The title compound was prepared from (R)-2-butyl-succinic acid 4-tert-butyl ester IV-a (R₂=n-butyl) and N-[1-((S)-2-amino-3,3-dimethyl-butyryl)-piperidin-4-yl]-4-methoxy-benzamide hydrochloride I-i (R₃=tert-butyl, n=0, R₅═R₆═R₈═R₉═H, R₇═OCH₃) according to General procedure IV.

¹H-NMR(CD₃OD): δ 7.79 (t, 2H), 6.96 (d, 2H), 4.88-4.94 (m, 1H), 4.53 (dd, 1H), 4.08-4.26 (m, 2H), 3.83 (s, 3H), 3.21-3.31 (m, 1H), 2.74-2.86 (m, 2H), 2.19-2.40 (m, 2H), 2.01 (t, 2H), 1.21-1.63 (m, 8H), 0.86-1.04 (m, 12H).

### EXAMPLE 20

### (R)-2-Cyclopentylmethyl-N¹-{(S)-2,2-dimethyl-1-[4-(4-methyl-benzoylamino)-piperidine-1-carbonyl]-propyl}-N⁴-hydroxy-succinamide

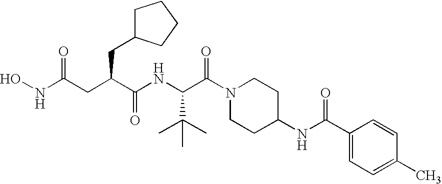

The title compound was prepared from (R)-2-cyclopentylmethyl-succinic acid 4-tert-butyl ester IV-a (R₂=cyclopentylmethyl) and N-[1-((S)-2-amino-3,3-dimethyl-butyryl)-piperidin-4-yl]-4methyl-benzamide hydrochloride I-i (R₃=tert-butyl, n=0, R₅═R₆═R₈═R₉═H, R₇═CH₃) according to General procedure IV.

¹H-NMR(DMSO-d₆): δ 7.74 (t, 2H), 7.23 (d, 2H), 4.80 (t, 1H), 4.29-4.42 (m, 2H), 4.00-4.10 (m, 2H), 3.13 (t, 1H), 2.68-2.80 (m, 2H), 2.33 (s, 3H), 1.97-2.12 (m, 2H), 1.08-1.81 (m, 13H), 0.93 (d, 11H).

### EXAMPLE 21

### (R)-2-Cyclopentylmethyl-N⁴-hydroxy-N¹-{(S)-1-[4-(4-methoxy-benzoylamino)-piperidine-1-carbonyl]-2,2-dimethyl-propyl}-succinamide

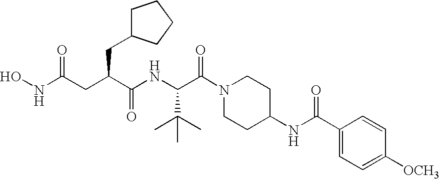

The title compound was prepared from (R)-2-cyclopentylmethyl-succinic acid 4-tert-butyl ester IV-a (R₂=cyclopentylmethyl) and N-[1-((S)-2-amino-3,3-dimethyl-butyryl)-piperidin-4-yl]4-methoxy-benzamide hydrochloride I-i (R₃=tertbutyl, n=0, R₅═R₆═R₈═R₉═H, R₇═OCH₃) according to General procedure IV.

¹H-NMR(DMSO-d₆): δ 7.81 (dd, 2H), 6.96 (dd, 2H), 4.80 (t, 1H), 4.29-4.42 (m, 1H), 4.00-4.10 (m, 2H), 3.78 (s, 3H), 3.13 (t, 1H), 2.67-2.80 (m, 2H), 1.97-2.12 (m, 2H), 1.13-1.81 (m, 13H), 0.93 (d, 11H, J=11.5 Hz).

### EXAMPLE 22

### N-[1-((S)-2-{(R)-2-[(Formyl-hydroxy-amino)-methyl]-hexanoylamino}-3,3-dimethyl-butyryl)-piperidin-4-yl)benzamide

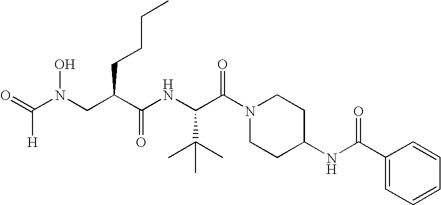

The title compound was prepared from (R)-2-[(benzyloxy-formyl-amino)-methyl]-hexanoic acid V-a (R₂=n-butyl) and N-[1-((S)-2-amino-3,3-dimethyl-butyryl)-piperidin-4yl]-benzamide hydrochloride I-i (R₃=tert-butyl, n=0, R₅═R₆═R₇═R₈═R₉═H) according to General procedure V.

¹H-NMR(CDCl₃): δ 8.35 (s, 0.3H), 7.75-7.82 (m, 2.7H), 7.40-7.52 (m, 3H), 4.84-4.96 (m, 1H), 4.59 (dd, 1H), 3.96-4.28 (m, 2H), 3.53-3.80 (m, 2H), 3.17-3.26 (m, 1H), 2.70-2.87 (m, 2H), 2.05-2.10 (m, 2H), 1.32-1.59 (m, 8H), 0.85-1.00 (m, 12H).

### EXAMPLE 23

### 4-Bromo-N-[1-((S)-2-{(R)-2-[(formyl-hydroxy-amino)-methyl)-hexanoylamino}-3,3-dimethyl-butyryl)-piperidin-4yl]benzamide

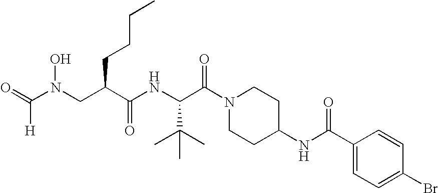

The title compound was prepared from (R)-2-[(benzyloxy-formyl-amino)-methyl]-hexanoic acid V-a (R₂=n-butyl) and N-[1S-((S)-2-amino-3,3-dimethyl-butyryl)-piperidin-4-yl]-4-bromo-benzamide hydrochloride I-i (R₃=tert-butyl, n=0, R₅═R₆═R₈═R₉═H, R₇═Br) according to General procedure V.

¹H-NMR(CDCl₃): δ 7.75-7.82 (m, 2H), 7.41-7.51 (m, 2H), 4.89-4.96 (m, 1H), 4.62 (dd, 1H), 4.21 (m, 2H), 3.46-3.73 (m, 1H), 3.11-3.30 (m, 2H), 2.59-2.85 (m, 2H), 2.05-2.11 (m, 2H), 1.29-1.58 (m, 8H), 0.85-1.01 (m, 12H).

### EXAMPLE 24

### (R)-2-[(Formyl-hydroxy-amino)-methyl)-hexanoic acid [(S)-2,2-dimethyl-1-(4-phenylacetylamino-piperidine-1-carbonyl)-propyl]-amide

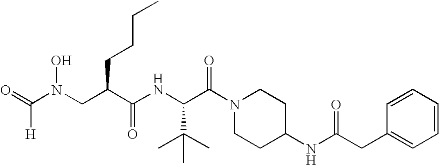

The title compound was prepared from (R)-2-[(benzyloxy-formyl-amino)-methyl]-hexanoic acid V-a (R₂=n-butyl) and N-[1-((S)-2-amino-3,3-dimethyl-butyryl)-piperidin-4-yl]-2-phenyl-acetamide hydrochloride I-i (R₃=tert-butyl, n=1, R₅═R₆═R₇═R₈═R₉═H) according to General procedure V.

¹H-NMR(CDCl₃): δ 8.25 (s, 0.3H), 7.77 (s, 0.7H), 7.22-7.36 (m, 5H), 4.88 (d, 1H), 4.44 (dd, 1H), 4.04 (m, 2H), 3.45-3.75 (m, 4H), 3.08-3.17 (m, 1H), 2.62-2.80 (m, 2H), 1.93 (m, 2H), 1.25-1.55 (m, 8H), 0.79-0.96 (m, 12H).

### EXAMPLE 25

### N-(1-{(S)-2-[(R)-2-Cyclopentylmethyl-3-(formyl-hydroxy-amino)-propionylamino]-3,3dimethyl-butyryl}-piperidin-4-yl)-benzamide

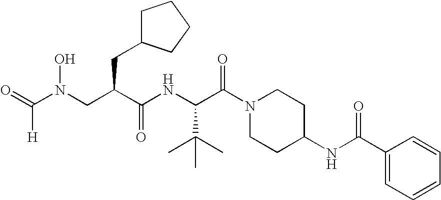

The title compound was prepared from (R)-3-(Benzyloxy-formyl-amino)-2cyclopentylmethyl-propionic acid V-a (R₂=cyclopentylmethyl) and N-[1-((S)-2-amino-3,3-dimethyl-butyryl)-piperidin-4-yl]-benzamide hydrochloride I-i (R₃=tert-butyl, n=0, R₅═R₆═R₇═R₈═R₉═H) according to General procedure V.

¹H-NMR(DMSO-d₆): δ 8.22 (s, 0.3H), 7.75-7.86 (m, 2.7H), 7.44-7.51 (m, 3H), 4.86 (t, 1H), 4.28-4.44 (m, 1H), 3.99-4.13 (m, 2H), 3.54-3.67 (m, 1H), 3.12-3.43 (m, 2H), 2.67-2.92 (m, 2H), 1.15-2.50 (m, 13H), 0.90-0.94 (m, 11H).

### EXAMPLE 26

### 4-Bromo-N-(1-{(S)-2-[(R)-2-cyclopentylmethyl-3-(formyl-hydroxy-amino)-propionylamino]-3,3dimethyl-4-butyryl}-piperidin-4-yl)-benzamide

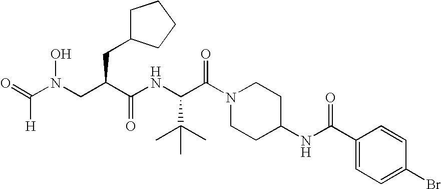

The title compound was prepared from (R)-3-(Benzyloxy-formyl-amino)-2-cyclopentylmethyl-propionic acid V-a (R₂=cyclopentylmethyl) and N-[1-((S)-2-amino-3,3-dimethyl-butyryl)-piperidin-4-yl]-4-bromo-benzamide hydrochloride I-i (R₃=tert-butyl, n=0, R₅═R₆═R₈═R₉═H, R₇═Br) according to General procedure V.

¹H-NMR(CDCl₃): δ 7.77 (d, 2H), 7.40-7.53 (m, 2H), 4.70 (br s, 1H), 4.43 (m, 1H), 4.08-4.27 (m, 2H), 3.80 (m, 1H), 2.74-3.16 (m, 2H), 2.04-2.18 (m, 4H), 1.43-1.75 (m, 11H), 0.94-1.07 (m, 11H).

### EXAMPLE 27

### (R)-2-Cyclopentylmethyl-N-[(S)-2,2-dimethyl-1-(4-phenylacetylamino-piperidine-1--carbonyl)-propyl]-3-(formyl-hydroxy-amino)-propionamide

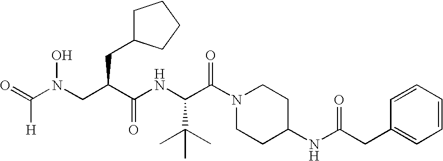

The title compound was prepared from (R)-3-(Benzyloxy-formyl-amino)-2cyclopentylmethyl-propionic acid V-a (R₂=cyclopentylmethyl) and N-[1-((S)-2-amino-3,3-dimethyl-butyryl)-piperidinyl]-4-yl]-2-phenyl-acetamide hydrochloride I-i (R₃=tert-butyl, n=1, R₅═R₆═R₇═R₈═R₉═H) according to General procedure V.

¹H-NMR(CDCl₃): δ 8.25 (s, 0.4H), 7.77 (s, 0.6H), 7.21-7.37 (m, 5H), 4.78-4.89 (m, 1H), 4.30-4.56 (m, 1H), 4.02 (m, 2H), 3.71-3.79 (m, 1H), 3.50-3.57 (m, 4H), 3.12 (m, 1H), 2.60-2.81 (m, 2H), 1.43-1.79 (m, 11H), 0.92-1.06 (m, 11H).

### EXAMPLE 28

### N-(1-{(S)-2-[(R)-2-Cyclopentylmethyl-3-(formyl-hydroxy-amino)-propionylamino]-3,3-dimethyl-4-butyryl}-piperidin-4-yl)-4-trifluoromethyl-benzamide

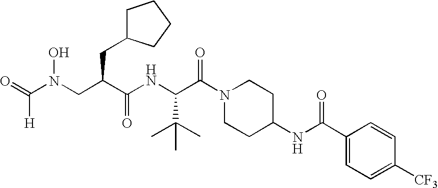

The title compound was prepared from (R)-3-(Benzyloxy-formyl-amino)-2-cyclopentylmethyl-propionic acid V-a (R₂=cyclopentylmethyl) and N-[1-((S)-2-amino-3,3-dimethyl-butyryl)-piperidin-4-yl]-4-trifluoromethyl-benzamide hydrochloride I-i (R₃=tert-butyl, n=0, R₅═R₆═R₈═R₉═H, R₇═CF₃) according to General procedure V.

¹H-NMR(CDCl₃): δ 8.32 (s, 0.3H), 7.86-7.93 (m, 2H), 7.78 (s, 0.7H), 7.68 (d, 2H), 4.82-4.93 (m, 1H), 4.61-4.65 (m, 1H), 3.98-4.31 (m, 3H), 3.58-3.73 (m, 1H), 3.21-3.25 (m, 1H), 2.63-2.90 (m, 2H), 2.04-2.18 (m, 3H), 1.26-1.82 (m, 10H), 0.97-1.11 (m, 11H).

### EXAMPLE 29

### 4-Cyano-N-(1-{(S)-2-[(R)-2-cyclopentylmethyl-3-(formyl-hydroxy-amino)-propionylamino]-3,3-dimethyl-butyryl}-piperidin-4-yl)benzamide

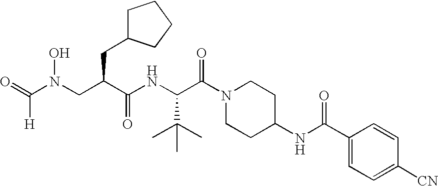

The title compound was prepared from (R)-3-(Benzyloxy-formyl-amino)-2-cyclopentylmethyl-propionic acid V-a (R₂=cyclopentylmethyl) and N-[1-{(S)-2-amino-3,3-dimethyl-butyryl)-piperidin-4-yl]-4-cyano-benzamide hydrochloride I-i (R₃=tert-butyl, n=0, R₅═R₆═R₈═R₉═H, R₇═CN) according to General procedure V.

¹H-NMR(CDCl₃): δ 8.35 (s, 0.3H), 7.80 (s, 0.7H), 7.64-7.73 (m, 2H), 7.21-7.27 (m, 2H), 4.83-4.96 (m, 1H), 4.61 (dd, 1H), 4.16 (m, 3H), 3.54-3.74 (m, 1H), 3.21 (m, 1H), 2.64-2.87 (m, 2H), 1.26-2.06 (m, 13H), 0.96-1.10 (m, 11H).

### EXAMPLE 30

### N-(1-{(S)-2-[(R)-2-Cyclopentylmethyl-3-(formyl-hydroxy-amino)-propionylamino]-3,3-dimethyl-butyryl}-piperidin-4-yl)-4-fluoro-benzamide

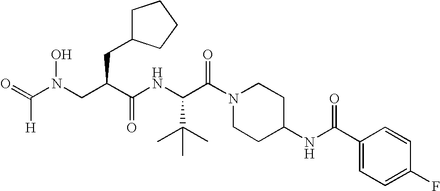

The title compound was prepared from (R)-3-(Benzyloxy-formyl-amino)-2-cyclopentylmethyl-propionic acid V-a (R₂=cyclopentylmethyl) and N-[1-((S)-2-amino-3,3-dimethyl-butyryl)-piperidin-4-yl]-4-fluoro-benzamide hydrochloride I-i (R₃=tert-butyl, n=0, R₅═R₆═R₈═R₉═H, R₇═F) according to General procedure V.

¹H-NMR(CDCl₃): δ 8.33 (s, 0.3H), 7.79-7.82 (m, 2.7H), 7.07-7.12 (m, 2H), 4.82-4.96 (m, 1H), 4.61-4.65 (m, 1H), 4.004.15 (m, 3H), 3.57-3.81 (m, 1H), 3.16-3.25 (m, 1H), 2.64-2.87 (m, 2H), 1.24-2.18 (m, 13H), 0.97-1.11 (m, 11H).

### EXAMPLE 31

### N-[1-((S)-2-{(R)-2-[(Formyl-hydroxy-amino)-methyl]-4-methyl-pentanoylamino}-3,3-dimethyl-butyryl)-piperidin-4-yl]benzamide

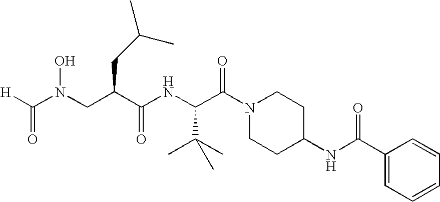

The title compound was prepared from (R)-2-[(Benzyloxy-formyl-amino)-methyl]-4-methyl-pentanoic acid V-a (R₂=iso-butyl) and N-[1-((S)-2-amino-3,3-dimethyl-butyryl)-piperidin-4-yl]-benzamide hydrochloride I-i (R₃=tert-butyl, n=0, R₅═R₆═R₇═R₈═R₉═H) according to General procedure V.

¹H-NMR(DMSO-d₆): δ 8.21 (s, 0.3H), 7,81-7.84 (m, 2H), 7.74 (s, 0.7H), 7.41-7.50 (m, 3H), 4.83 (m, 1H), 4.34 (dd, 1H), 4.11 (m, 2H), 3.54-3.61 (m, 1H), 3.11-3.19 (m, 2H), 2.60-3.00 (m, 2H), 1.81-1.86 (m, 2H), 1.24-1.41 (m, 4H), 1.10 (m, 1H), 0.82-0.93 (m, 15H).

### EXAMPLE 32

### N-[1-((S)-2-{(R)-2-[(Formyl-hydroxy-amino)-methyl]methyl-pentanoylamino}-3,3-dimethyl-butyryl)-piperidin-4-yl]-4-trifluoromethyl-benzamide

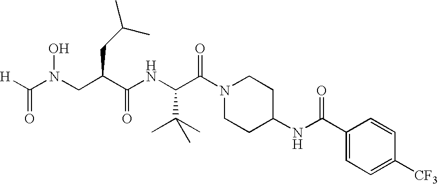

The title compound was prepared from (R)-2-[(Benzyloxy-formyl-amino)-methyl]-4-methyl-pentanoic acid V-a (R₂=iso-butyl) and N-[1-((S)-2-amino-3,3-dimethyl-butyryl)-piperidin-4-yl]-4-trifluoromethyl-benzamide hydrochloride I-i (R₃=tert-butyl, n=0, R₅═R₆═R₈═R₉═H, R₇═CF₃) according to General procedure V.

¹H-NMR(DMSO-d₆): δ 8.21 (s, 0.4H), 8.02 (dd, 2H), 7.83 (d, 2H), 7.74 (s, 0.6H), 4.83 (t, 1H), 4.35 (dd, 1H), 4.12 (m, 2H), 3.54-3.61 (m, 1H), 3.12-3.32 (m, 2H), 2.60-3.00 (m, 2H), 1.84-1.98 (m, 2H), 1.00-1.44 (m, 5H), 0.78-0.93 (m, 15H).

### EXAMPLE 33

### 4-Cyano-N-[1-((S)-2-{(R)-2-[(formyl-hydroxy-amino)-methyl]-4-methyl-pentanoylamino}-3,3-dimethyl-butyryl)-piperidin-4-yl]benzamide

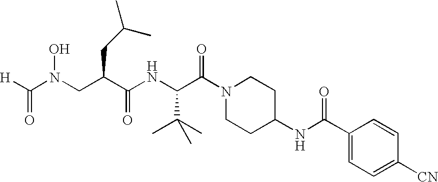

The title compound was prepared from (R)-2-[(Benzyloxy-formyl-amino)-methyl]-4-methyl-pentanoic acid V-a (R₂=iso-butyl) and N-[1-((S)-2-amino-3,3-dimethyl-butyryl)-piperidin-4-yl]-4-cyano-benzamide hydrochloride I-i (R₃=tert-butyl, n=0, R₅═R₆═R₈═R₉═H, R₇═CN) according to General procedure V.

¹H-NMR(DMSO-d₆): δ 8.21 (s, 0.2H), 7.74 (s, 0.8H), 7.74-8.00 (m, 5H), 4.82 (t, 1H), 4.35 (dd, 1H), 4.11 (m, 2H), 3.53-3.69 (m, 1H), 3.16-3.36 (m, 2H), 2.60-3.00 (m, 2H), 1.83-1.93 (m, 2H), 1.06-1.41 (m, 5H), 0.78-0.92 (m, 15H).

### EXAMPLE 34

### 4-Fluoro-N-[1-((S)-2-{(R)-2-[(formyl-hydroxy-amino)-methyl]-hexanoylamino)-3,3-dimethyl-butyryl)-piperidin-4-yl]benzamide

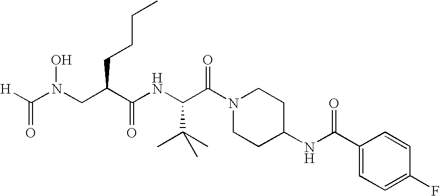

The title compound was prepared from (R)-2-[(benzyloxy-formyl-amino)-methyl]-hexanoic acid V-a (R₂=n-butyl) and N-[1-((S)-2-amino-3,3-dimethyl-butyryl)-piperidin-4-yl]-4-fluoro-benzamide hydrochloride I-i (R₃=tert-butyl, n=0, R₅═R₆═R₈═R₉═H, R₈═F) according to General procedure V.

¹H-NMR(DMSO-d₆): δ 8.21 (s, 0.3H), 7.88-7.93 (m, 2H), 7.75 (s, 0.7H), 7.27 (dt, 2H ), 4.83 (t, 1H), 4.38 (m, 1H), 4.00-4.13 (m, 2H), 3.49-3.58 (m, 1H), 3.11-3.33 (m, 2H), 2.71-3.00 (m, 2H), 1.83 (m, 2H), 1.18 (m, 8H), 0.81-0.93 (m, 12H).

### EXAMPLE 35

### 4-Fluoro-N-[1-((S)-2-{(R)-2-[(formyl-hydroxy-amino)-methyl]-4-methyl-pentanoylamino}-3,3-dimethyl-butyryl)-piperidin-4-yl]benzamide

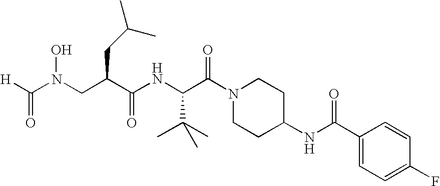

The title compound was prepared from (R)-2-[(Benzyloxy-formyl-amino)-methyl]-4-methyl-pentanoic acid V-a (R₂=iso-butyl) and N-[1-((S)-2-amino-3,3-dimethyl-butyryl)-piperidin-4-yl]-4-fluorobenzamide hydrochloride I-i (R₃=tert-butyl, n=0, R₅═R₆═R₈═R₉═H, R₇═F) according to General procedure V.

¹H-NMR(DMSO-d₆): δ 8.21 (s, 0.3H), 7.88-7.92 (m, 2H), 7.74 (s, 0.7H), 7.27 (dt, 2H), 4.82 (t, 1H), 4.33 (dd, 1H), 4.01-4.12 (m, 2H), 3.54-3.61 (m, 1H), 3.15-3.36 (m, 2H), 2.70-3.00 (m, 2H), 1.82-1.94 (m, 2H), 1.41 (m, 4H), 1.08-1.19 (m, 1H), 0.81-0.93 (m, 15H).

### EXAMPLE 36

### N-[1 -((S)-2-{(R)-2-[(Formyl-hydroxy-amino)-methyl]-hexanoylamino}-3,3-dimethyl-butyryl)-piperidin-4-yl]-4-methyl-benzamide

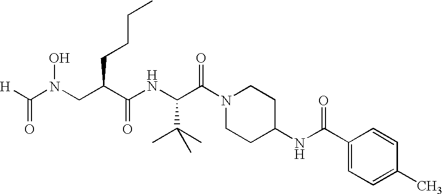

The title compound was prepared from (R)-2-[(benzyloxy-formyl-amino)-methyl]-hexanoic acid V-a (R₂=n-butyl) and N-[1-((S)-2-amino-3,3-dimethyl-butyryl)-piperidin-4-yl]-4-methyl-benzamide hydrochloride I-i (R₃=tert-butyl, n=0, R₅═R₆═R₈═R₉═H, R₇═CH₃) according to General procedure V.

¹H-NMR(DMSO-d₆): δ 8.21 (s, 0.3H), 7.76 (s, 0.7H), 7.74 (dd, 2H), 7.24 (dd, 2H), 4.83 (t, 1H), 4.31-4.42 (m, 1H), 3.98-4.15 (m, 2H), 3.49-3.63 (m, 1H), 3.10-3.33 (m, 2H), 2.66-2.90 (m, 2H), 2.33 (s, 3H), 1.82-1.85 (m, 2H), 1.14-1.38 (m, 8H), 0.81-0.93 (m, 12H).

### EXAMPLE 37

### N-[1-((S)-2-{(R)-2-[(Formyl-hydroxy-amino)-methyl)-hexanoylamino}-3,3-dimethyl-butyryl)-piperidin-4-yl]-4-methoxy-benzamide

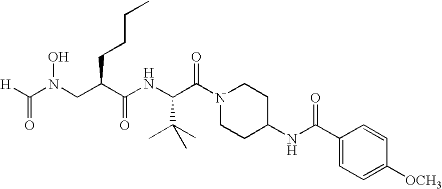

The title compound was prepared from (R)-2-[(benzyloxy-formyl-amino)-methyl]-hexanoic acid V-a (R₂=n-butyl) and N-[1-((S)-2-amino-3,3-dimethyl-butyryl)-piperidin-4-yl]-4-methoxy-benzamide hydrochloride I-i (R₃=tert-butyl, n=0, R₅═R₆═R₈═R₉═H, R═OCH₃) according to General procedure V.

¹H-NMR(DMSO-d₆): δ 8.21 (s, 0.3H), 7.81 (dd, 2H), 7.75 (s, 0.7H), 6.96 (dd, 2H), 4.83 (t, 1H), 4.31-4.42 (m, 1H), 3.98-4.11 (m, 2H), 3.49-3.58 (m, 1H), 3.33 (s, 3H), 3.10-3.37 (m, 2H), 2.66-2.88 (m, 2H), 1.81-1.85 (m, 2H), 1.14-1.38 (m, 8H), 0.81-0.93 (m, 12H).

### EXAMPLE 38

### N-(1-[(S)-2-[(R)-2-Cyclopentylmethyl-3-(formyl-hydroxy-amino)-propionylamino]-3,3-dimethyl-butyryl}-piperidin-4-yl)-4-methyl-benzamide

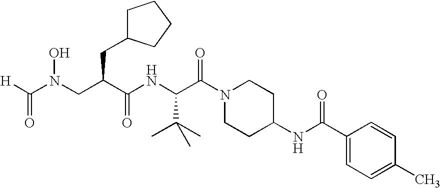

The title compound was prepared from (R)-3-(Benzyloxy-formyl-amino)-2-cyclopentylmethyl-propionic acid V-a (R₂=cyclopentylmethyl) and N-[1-((S)-2-amino-3,3-dimethyl-butyryl)-piperidin-4-yl]-4-methyl-benzamide hydrochloride I-i (R₃=tert-butyl, n=0, R₅═R₆═R₈═R₉═H, R₇═CH₃) according to General procedure V.

¹H-NMR(DMSO-d₆): δ 8.21 (s, 0.4H), 7.74 (s, 0.6H), 7.71-7.76 (m, 2H), 7.24 (dd, 2H), 4.85 (t, 1H), 4.35 (dd, 1H), 3.98-4.12 (m, 2H), 3.53-3.66 (m, 1H), 3.10-3.43 (m, 2H), 2.65-2.91 (m, 2H), 2.33 (s, 3H), 1.14-1.85 (m, 13H), 0.89-0.92 (m, 11H).

### EXAMPLE 39

### N-(1-{(S)-2-[(R)-2-Cyclopentylmethyl-3-(formyl-hydroxy-amino)-propionylamino]-3,3-dimethyl-butyryl}-piperidin-4-yl)-4-methoxy-benzamide

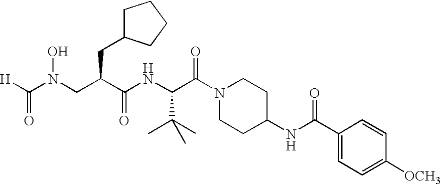

The title compound was prepared from (R)-3-(Benzyloxy-formyl-amino)-2-cyclopentylmethyl-propionic acid V-a (R₂=cyclopentylmethyl) and N-[1-((S)-2-amino-3,3-dimethyl-butyryl)-piperidin-4-yl]-4-methoxy-benzamide hydrochloride I-i (R₃=tert-butyl, n=0, R₅═R₆═R₈═R₉═H, R₇═OCH₃) according to General procedure V.

¹H-NMR(DMSO-d₆): δ 8.21 (s, 0.4H), 7.74 (s, 0.6H), 7.81 (dd, 2H), 6.96 (dd, 2H), 4.85 (t, 1H), 4.35 (dd, 1H), 3.98-4.12 (m, 2H), 3.79 (s, 3H), 3.57 (m, 1H), 3.09-3.33 (m, 2H), 2.65-3.00 (m, 2H), 1.14-1.85 (m, 13H), 0.89-0.93 (m, 11H).

### EXAMPLE 40

### N-(1-{(S)-2-[(R)-2-Cyclopentylmethyl-3-(formyl-hydroxy-amino)-propionyl-amino]-3-phenyl-propionyl}-piperidin-4-yl)-4-fluoro-benzamide

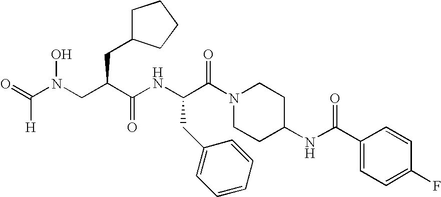

The title compound was prepared from (R)-3-(Benzyloxy-formyl-amino)-2-cyclopentylmethyl-propionic acid V-a (R₂=cyclopentylmethyl) and N-[1-((S)-2-amino-3-phenyl-propionyl)-piperidin-4-yl]-4-fluorobenzamide hydrochloride I-i (R₃=benzyl, n=0, R₅═R₆═R₈═R₉═H, R₇═F) according to General procedure V.

¹H-NMR(CDCl₃): δ 8.33 (s, 0.3H), 7.79-7.82 (m, 2.7H), 7.35-6.95 (m, 7H), 4.82-4.96 (m, 1H), 4.61-4.65 (m, 1H), 4.00-4.15 (m, 3H), 3.57-3.81 (m, 1H), 3.16-3.25 (m, 1H), 3.11-2.91(m, 2H), 2.64-2.87 (m, 2H), 1.24-2.18 (m, 13H), 0.97-1.11 (m, 2H).

### EXAMPLE 41

### 4-Cyano-N-(1-{(S)-2-[(R)-2-cyclopentylmethyl-3-(formyl-hydroxy-amino)-propionylamino]-3-methyl-butyryl}-piperidin-4-yl)benzamide

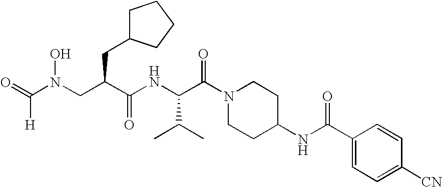

The title compound was prepared from (R)-3-(Benzyloxy-formyl-amino)-2-cyclopentylmethyl-propionic acid V-a (R₂=cyclopentylmethyl) and N-[1-((S)-2-amino-3-methyl-butyryl)-piperidin-4-yl]-4-cyano-benzamide hydrochloride I-i (R₃=iso-propyl, n=0, R₅═R₆═R₈═R₉═H, R₇═CN) according to General procedure V.

¹H-NMR(CDCl₃): δ 8.35 (s, 0.3H), 7.80 (s, 0.7H), 7.64-7.73 (m, 2H), 7.21-7.27 (m, 2H), 4.83-4.96 (m, 1H), 4.61 (dd, 1H), 4.16 (m, 3H), 3.54-3.74 (m, 1H), 3.21 (m, 1H), 2.64-2.87 (m, 2H), 1.26-2.06 (m, 13H), 0.96-1.10 (m, 8H).

### EXAMPLE 42

### N-(1-{(S)-2-[(R)-2-Cyclopentylmethyl-3-(formyl-hydroxy-amino)-propionylamino]-2-phenyl-acetyl}-piperidin-4-yl)-4-trifluoromethyl-benzamide

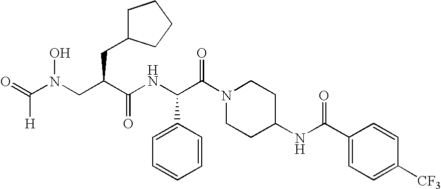

The title compound was prepared from (R)-3-(Benzyloxy-formyl-amino)-2-cyclopentylmethyl-propionic acid V-a (R₂=cyclopentylmethyl) and N-[1-((S)-2-amino-2-phenyl-acetyl)-piperidin-4-yl]-4-trifluoromethyl-benzamide hydrochloride I-i (R₃=phenyl, n=0, R₅═R₆═R₈═R₉═H, R₇═CF₃) according to General procedure V.

¹H-NMR(CDCl₃): δ 8.32 (s, 0.3H), 7.86-7.93 (m, 2H), 7.78 (s, 0.7H), 7.68 (d, 2H), 4.82-4.93 (m, 1H), 4.61-4.65 (m, 1H), 3.98-4.31 (m, 3H), 3.58-3.73 (m, 1H), 3.21-3.25 (m, 1H), 2.63-2.90 (m, 2H), 2.04-2.18 (m, 3H), 1.26-1.82 (m, 10H), 0.97-1.11 (m, 2H).

### EXAMPLE 43

### N-(1-{(S)-2-[(R)-2-Cyclopentylmethyl-3-(formyl-hydroxy-amino)-propionylamino]-3,3-dimethyl-butyryl}-piperidin-4-yl)-2,4-difluorobenzamide

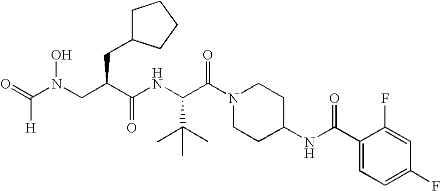

The title compound was prepared from (R)-3-(Benzyloxy-formyl-amino)-2-cyclopentylmethyl-propionic acid V-a (R₂=cyclopentylmethyl) and N-[1-((S)-2-amino-3,3-dimethyl-butyryl)-piperidin-4-yl]-2,4-difluorobenzamide hydrochloride I-i (R₃=tert-butyl, n=0, R₆═R₈═R₉═H, R₅═R₇═F) according to General procedure V.

¹H-NMR(CDCl₃): δ 8.33 (s, 0.3H), 7.81-7.92 (m, 1.7H), 6.99-7.12 (m, 2H), 4.82-4.96 (m, 1H), 4.61-4.65 (m, 1H), 4.00-4.15 (m, 3H), 3.57-3.81 (m, 1H), 3.16-3.25 (m, 1H), 2.64-2.87 (m, 2H), 1.24-2.18 (m, 13H), 0.97-1.11 (m, 11H).

### EXAMPLE 44

### N-(1-{(S)-2-[(R)-2-Cyclopentylmethyl-3-(formyl-hydroxy-amino)-propionylamino]-3,3-dimethyl-butyryl}-piperidin-4-yl)-2,4,5-trifluoro-benzamide

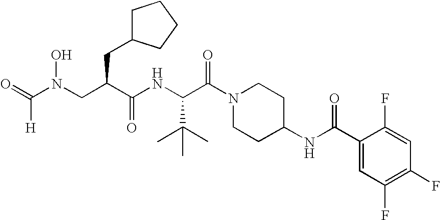

The title compound was prepared from (R)-3-(Benzyloxy-formyl-amino)-2-cyclopentylmethyl-propionic acid V-a (R₂=cyclopentylmethyl) and N-[1-((S)-2-amino-3,3-dimethyl-butyryl)-piperidin-4-yl]-2,4,5-trifluoro-benzamide hydrochloride I-i (R₃=tert-butyl, n=0, R₆═R₉═H, R₅═R₇═R₈═F) according to General procedure V.

¹H-NMR(CDCl₃): δ 8.32 (s, 0.3H), 7.85 (s, 0.7H), 7.69-7.80 (m, 1H), 6.98-7.12 (m, 1H), 4.82-4.96 (m, 1H), 4.61-4.65 (m, 1H), 4.00-4.15 (m, 3H), 3.57-3.81 (m, 1H), 3.16-3.25 (m, 1H), 2.64-2.87 (m, 2H), 1.24-2.18 (m, 13H), 0.97-1.11 (m, 11H).

### EXAMPLE 45

### N-(1-{(S)-2-[(R)-2-Cyclopentylmethyl-3-(formyl-hydroxy-amino)-propionylamino]-3,3-dimethyl-butyryl}-piperidin-4-yl)-3,4,5-trimethoxy-benzamide

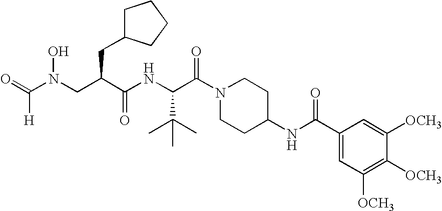

The title compound was prepared from (R)-3-(Benzyloxy-formyl-amino)-2-cyclopentylmethyl-propionic acid V-a (R₂=cyclopentylmethyl) and N-[1-((S)-2-amino-3,3-dimethyl-butyryl)piperidin-4-yl]-3,4,5-trimethoxy-benzamide hydrochloride I-i (R₃=tert-butyl, n=0, R₅═R₉═H, R₆═R₇═R₈═OCH₃) according to General procedure V.

¹H-NMR(CDCl₃): δ 8.21 (s, 0.4H), 7.74 (s, 0.6H), 7.11-6.96 (dd, 2H), 4.84 (t, 1H), 4.35 (dd, 1H), 3.99-4.11 (m, 2H), 3.78 (s, 3H), 3.57 (m, 1H), 3.08-3.33 (m, 2H), 2.64-3.00 (m, 2H), 1.14-1.85 (m, 13H), 0.89-0.93 (m, 11H).

### EXAMPLE 46

### (R)-2-Cyclopentylmethyl-N-((S)-1-{4-[3-(4-fluoro-phenyl)-ureido]-piperidine-1-carbonyl}-2,2-dimethyl-propyl)-3-(formyl-hydroxy-amino)-propionamide

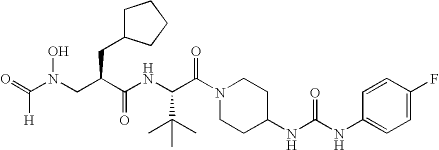

The title compound was prepared from (R)-3-(Benzyloxy-formyl-amino)-2-cyclopentylmethyl-propionic acid V-a (R₂=cyclopentylmethyl) and 1-[1-((S)-2-amino-3,3-dimethyl-butyryl)-piperidin-4-yl]-3-(4-fluoro-phenyl)-urea hydrochloride II-e (R₃=tert-butyl, n=0, R₅═R₆═R₈═R₉═H, R₇═F) according to General procedure V.

¹H-NMR(CDCl₃): δ 8.31 (s, 0.3H), 7.79-7.81 (m, 2.7H), 7.06-7.12 (m, 2H), 4.82-4.94 (m, 1H), 4.60-4.65 (m, 1H), 4.00-4.14 (m, 3H), 3.56-3.81 (m, 1H), 3.15-3.25 (m, 1H), 2.64-2.87 (m, 2H), 1.23-2.18 (m, 13H), 0.97-1.10 (m, 11H).

### EXAMPLE 47

### (R)-2-Cyclopentylmethyl-N-{(S)-2,2-dimethyl-1-[4-(3-p-tolyl-ureido)-piperidine-1-carbonyl]-propyl}-3-(formyl-hydroxy-amino)-propionamide

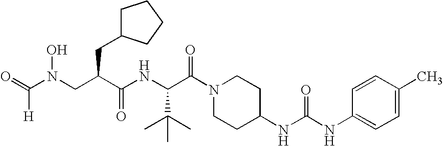

The title compound was prepared from (R)-3-(Benzyloxy-formyl-amino)-2-cyclopentylmethyl-propionic acid V-a (R₂=cyclopentylmethyl) and 1-[1-((S)-2-amino-3,3-dimethyl-butyryl)-piperidin-4-yl)-3-p-toyl-urea hydrochloride II-e (R₃=tert-butyl, n=0, R₅═R₆═R₈═R₉═H, R₇═CH₃) according to General procedure V.

¹H-NMR(CDCl₃): δ 8.20 (s, 0.4H), 7.73 (s, 0.6H), 7.70-7.75 (m, 2H), 7.24 (dd, 2H), 4.85 (t, 1H), 4.34 (dd, 1H), 3.96-4.11 (m, 2H), 3.53-3.66 (m, 1H), 3.11-3.43 (m, 2H), 2.66-2.91 (m, 2H), 2.32 (s, 3H), 1.14-1.84 (m, 13H), 0.89-0.91 (m, 11H).

### EXAMPLE 48

### (R)-2-Cyclopentylmethyl-N-((S)-2,2-dimethyl-1-{4-[3-(4-trifluoromethyl-phenyl)-ureido)-piperidine-1-carbonyl}-propyl)-3-(formyl-hydroxy-amino)-propionamide

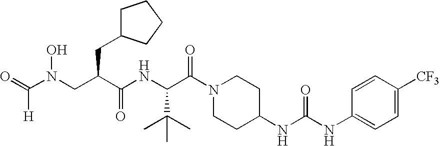

The title compound was prepared from (R)-3-(Benzyloxy-formyl-amino)-2cyclopentylmethyl-propionic acid V-a (R₂=cyclopentylmethyl) and 1-[1-((S)-2-am-no-3,3-dimethyl-butyryl)-piperidin-4-yl]-3-(4-trifluoromethyl-phenyl)urea hydrochloride II-e (R₃=tert-butyl, n=0, R₅═R₆═R₈═R₉=H, R₇=CF₃) according to General procedure V.

¹H-NMR(CDCl₃): δ 8.30 (s, 0.3H), 7.88-7.93 (m, 2H), 7.76 (s, 0.7H), 7.62 (d, 2H), 4.81-4.93 (m, 1H), 4.60-4.64 (m, 1H), 3.99-4.31 (m, 3H), 3.56-3.73 (m, 1H), 3.20-3.25 (m, 1H), 2.62-2.90 (m, 2H), 2.03-2.17 (m, 3H), 1.25-1.82 (m, 10H), 0.95-1.11 (m, 11H).

### EXAMPLE 49

### (R)-2-Cyclopentylmethyl-N-{(S)-1-[4-(2,4-difluoro-benzenesulfonylamino)-piperidine-1-carbonyl]-2,2-dimethyl-propyl}-3-(formyl-hydroxy-amino)-propionamide

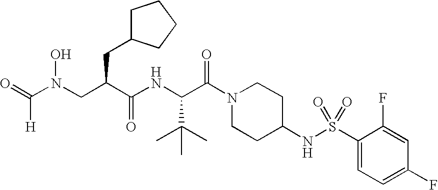

The title compound was prepared from (R)-3-(Benzyloxy-formyl-amino)-2-cyclopentylmethyl-propionic acid V-a (R₂=cyclopentylmethyl) and N-[1-((S)-2-amino-3,3-dimethyl-butyryl)-piperidin-4-yl]-2,4-difluoro-benzenesulfonamide hydrochloride III-e (R₃=tert-butyl, n=0, R₆═R₈═R₉═H, R₅═R₇═F) according to General procedure V.

¹H-NMR(CDCl₃): δ 8.31 (s, 0.3H), 7.80-7.94 (m, 1.7H), 6.99-7.10 (m, 2H), 4.83-4.95 (m, 1H), 4.60-4.63 (m, 1H), 3.99-4.14 (m, 3H), 3.56-3.81 (m, 1H), 3.15-3.25 (m, 1H), 2.63-2.87 (m, 2H), 1.23-2.18 (m, 13H), 0.96-1.10 (m, 11H).

### EXAMPLE 50

### (R)-2-Cyclopentylmethyl-N-{(S)-2,2-dimethyl-1-[4-(4-trifluoromethyl-benzenesulfonylamino)-piperidine-1-carbonyl]-propyl}-3-(formyl-hydroxy-amino)-propionamide

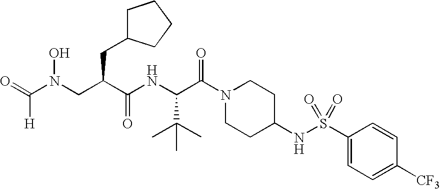

The title compound was prepared from (R)-3-(Benzyloxy-formyl-amino)-2-cyclopentylmethyl-propionic acid V-a (R₂=cyclopentylmethyl) and N-[1-((S)-2-amino-3,3-dimethyl-butyryl)-piperidin-4-yl]-4-trifluoromethyl-benzenesulfonamide hydrochloride III-e (R₃=tert-butyl, n=0, R₅═R₆═R₈═R₉═H, R₇═CF₃) according to General procedure V.

¹H-NMR(CDCl₃): δ 8.34 (s, 0.3H), 7.84-7.91 (m, 2H), 7.77 (s, 0.7H), 7.67 (d, 2H), 4.83-4.93 (m, 1H), 4.59-4.65 (m, 1H), 3.96-4.31 (m, 3H), 3.57-3.70 (m, 1H), 3.20-3.25 (m, 1H), 2.61-2.89 (m, 2H), 2.02-2.18 (m, 3H), 1.24-1.82 (m, 10H), 0.99-1.12 (m, 11H).

### EXAMPLE 51

### (R)-2-Cyclopentylmethyl-N-{(S)-2,2dimethyl-1-[4-(toluene-4-sulfonylamino)-piperidine-1-carbonyl]-propyl}-3-(formyl-hydroxy-amino)-propionamide

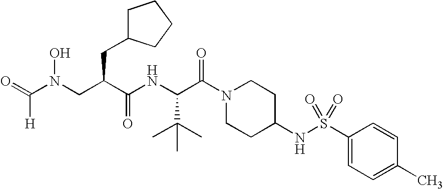

The title compound was prepared from (R)-3-(Benzyloxy-formyl-amino)-2-cyclopentylmethyl-propionic acid V-a (R₂=cyclopentylmethyl) and N-[1-((S)-2-amino-3,3dimethyl-butyryl)-piperidin-4-yl]-4-methyl-benzenesulfonamide hydrochloride III-e (R₃=tert-butyl, n=0, R₅═R₆═R₈═R₉═H, R₇═CH) according to General procedure V.

¹H-NMR(CDCl₃): δ 8.19 (s, 0.4H), 7.73 (s, 0.6H), 7.69-7.77 (m, 2H), 7.21 (dd, 2H), 4.81 (t, 1H), 4.31 (dd, 1H), 3.88-4.10 (m, 2H), 3.52-3.65 (m, 1H), 3.11-3.43 (m, 2H), 2.64-2.91 (m, 2H), 2.31 (s, 3H), 1.11-1.83 (m, 13H), 0.88-0.91 (m, 11H).

The present invention relates to antibacterial composition comprising a therapeutically effective amount of the compound of formula (I) or a pharmaceutically acceptable salt thereof and a pharmaceutically acceptable carrier. The compounds of this invention may be used to treat a subject to treat, prevent, and/or reduce the severity of an infection.

The present compounds are useful for the treatment of bacterial infection. Therefore, the pharmaceutical compositions of this invention may be administered in standard manner for the disease condition that it is desired to treat, for example by oral, rectal or parenteral administration. For these purposes the compounds of this invention may be formulated by means known in the art in to the form of, for example, tablets, capsules, aqueous or oily solutions or suspensions, (lipid) emulsions, dispersible powders, suppositories, ointments, creams, aerosols (or sprays), drops and sterile injectable aqueous or oily solutions or suspensions.

Compounds of formula (I) and their pharmaceutically acceptable salts which are active when given orally can be formulated as syrups, tablets, capsules, creams and lozenges.

Tablets and capsules for oral administration may be in unit dose presentation form, and may contain conventional excipients, disintegrants or glydents. For example, they may be syrup, gelatin, sorbitol, lactose, sugar, maize-starch, calcium phosphate, tabletting lubricant, magnesium stearate, polyethylene glycol, potato starch or sodium lauryl sulfate, and, if desired, conventional flavoring or coloring agent.

Typical parenteral compositions consist of a solution or suspension of a compound or salt in a sterile aqueous or non-aqueous carrier optionally containing a parenterally acceptably oil, for example polyethylene glycol, polyvinylpyrrolidone, lecithin, arachis oil or sesame oil.

Each dosage unit for oral administration contains preferably from 1 mg to 100 mg/Kg, and each dosage unit for parenteral administration contains suitably from 0.1 mg to 100 mg/Kg, of a compound of formula (I) or a pharmaceutically acceptable salt thereof calculated as the free acid.

The compounds of this invention, e.g. of formula (I) or a pharmaceutically acceptable salt thereof, may be administered alone or in combination with another therapeutic agent. Examples of such therapeutic agents include, but are not limited to, penicillins, cephalosporins, carbapenems, fluoroquinolones, clarithromycin, vancomycin, rifamycins, monebactams, licosamides, fosfomycin, glycopeptides, tetracyclines, streptogramins, chloramphenicol, oxazolidinone, corticosteroids, NSAID, narcotic or non-narcotic analgesics.

### EXPERIMENTAL EXAMPLE

### 1. FORMULATION EXAMPLES

The following are representative pharmaceutical formulations containing a compound of formula (I).

### EXAMPLE 1

### Tablet Formulation

The following ingredients are mixed and compressed into tablets using suitable punches.

TABLE 1

| Ingredient | Quantity |
|---|---|
| Compound of this invention | 400.0 mg |
| Cornstarch | 40.0 mg |
| Microcrystalline cellulose | 10.0 mg |
| Croscarmellose sodium | 25 mg |
| Lactose | 110 mg |
| Magnesium stearate | 5 mg |

### EXAMPLE 2

### Capsule Formulation

The following ingredients are mixed and filled into hard gelatin capsules of a suitable size.

TABLE 2

| Ingredient | Quantity |
|---|---|
| Compound of this invention | 250 mg |
| Lactose | 148 mg |
| Magnesium stearate | 2 mg |

### EXAMPLE 3

### Injectable Formulation

The following ingredients are mixed and filled into ampoules of a suitable size.

TABLE 3

| Ingredient | Quantity |
|---|---|
| Compound of this invention | 0.2 mg-20 mg |
| Sodium acetate buffer solution, 0.4 M | 20 mL |
| HCl (1N) or NaOH (1N) | q.s. to suitable pH |
| Water for injection | q.s. to 20 mL |

### 2. TEST FOR ANTIBACTERIAL ACTIVITY

Minimum inhibitory concentrations (MICs) were determined using the microdilution method in 96-well format plates. Each of the compounds of Examples was dissolved in dimethyl sulfoxide to a concentration of 2 mg/mL and stored at 4° C. until used. They were diluted in Mueller-Hinton Broth (MHB) and used for MIC determination. The range of concentrations tested was 64-0.00625 μg/mL final concentration using a two-fold dilution system. Plates were incubated at 37° C. and MIC were recorded after 24 hours of incubation for bacteria. MIC was defined as the lowest concentration of compound that does not produce visible growth after incubation.

Linezolid and vancomycin were used as standard antibiotics, respectively.

Results for some of the compounds of the Examples are reported in Table 4.

TABLE 4

| | Compds | | | | |
|---|---|---|---|---|---|
| | MIC (μg/mL) | | | | |
| | Methicillin-resistant | Vancomycin-resistant | | | |
| | Staphylococcus | Enterococcus | Streptococcus | Streptococcus | Streptococcus |
| | aureus | faecalis | pneumoniae | pyogenes | agalactiae |
| Example 8 | 12.5 | 25 | 6.3 | 1.6 | 1.6 |
| Example 10 | 50 | 50 | 50 | 0.8 | 0.2 |
| Example 13 | 12.5 | 6.3 | 3.1 | 0.4 | 0.2 |
| Example 15 | 6.3 | 6.3 | 6.3 | 0.2 | 0.2 |
| Example 20 | 25 | 12.5 | 0.8 | 0.4 | 0.4 |
| Example 25 | 12.5 | 6.3 | 6.3 | 0.4 | 0.1 |
| Example 28 | 3.1 | 6.3 | 1.6 | 3.1 | 0.1 |
| Example 29 | 3.1 | 3.1 | 1.6 | 0.1 | 0.1 |
| Example 30 | 6.3 | 6.3 | 1.6 | 0.2 | 0.2 |
| Example 38 | 6.3 | 3.1 | 1.6 | 3.1 | 0.1 |
| Example 39 | 12.5 | 6.3 | 3.1 | 0.2 | 0.2 |
| Example 41 | 6.3 | 3.1 | 1.6 | 0.4 | 0.4 |
| Example 44 | 3.1 | 3.1 | 1.6 | 0.2 | 0.2 |
| Example 48 | 6.3 | 3.1 | 1.6 | 0.2 | 0.2 |
| Example 50 | 6.3 | 3.1 | 1.6 | 0.4 | 0.4 |
| Linezolid | 1.6 | 0.8 | 0.8 | 0.8 | 1.6 |
| Vancomycin | 3.1 | >100 | 0.8 | 0.8 | 1.6 |

### 3. ACUTE TOXICITY

To demonstrate the usefulness of the compounds of this invention as medicaments we have performed acute toxicity study in mice.

The acute toxicity of the compounds of Example 7, 13, 18 and 47 were tested using several groups of ICR mice each of 6 mice. 4,000 mg/kg dose of the medicament was each orally injected into each group of mice, and weight change and death were Observed for 14 days after the injection.

The LD₅₀ values obtained in mice for the compounds of this invention are summarized in Table 5.

TABLE 5

| Compds | LD₅₀ (mg/kg) |
|---|---|
| Example 7 | >4,000 |
| Example 13 | >4,000 |
| Example 18 | >4,000 |
| Example 47 | >4,000 |

animal: ICR mice (♂, 4 weeks)

### INDUSTRIAL APPLICABILITY

The compounds of this invention, e.g. of formula (I) or a pharmaceutically acceptable salt thereof have low toxicity and are antibacterially active against gram-positive organisms, in particular also against those microorganisms which are resistant to various antibiotics. Thus, the compounds of this invention are useful as antibacterial agents for infection with resistant bacteria.

1. A compound of formula (I) or a pharmaceutically acceptable salts thereof: 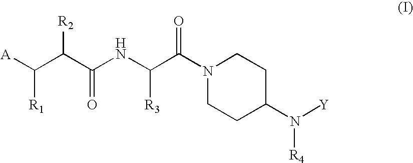
wherein, A is selected from the group of consisting of —C(═O)NHOH or —N(CHO)OH;
R₁ represents hydrogen, C₁₋₃ alkyl, C₄₋₆ cycloalkyl, halogen or hydroxy group;
R₂ represents hydrogen, straight or branched C₁₋₆ alkyl, straight or branched C₁₋₆ alkenyl, C₄₋₆ cycloalkyl, C₄₋₆ heterocycle including nitrogen or oxygen, or benzyl group;
R₃ represents hydrogen, methyl, straight or branched C₁₋₆ alkyl, straight or branched C₁₋₆ alkenyl, C₄₋₆ cycloalkyl, phenyl or benzyl group;
R₄ represents hydrogen, straight or branched C₁₋₄ alkyl, C₁₋₄ alkenyl, hydroxy substituted C₄₋₆ cycloalkyl group; and
Y represents a group of formula (IIa), or (IIb), or (IIc): 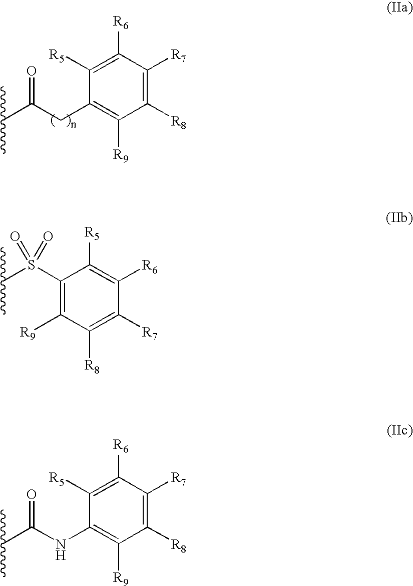
wherein, n is independently 0 or 1;
each of R₅, R₆, R₇, R₈ and R₉ is independently hydrogen, straight or branched C₁₋₃ alkyl, hydroxy, alkoxy, acyl, acyloxy, halogen (fluoro, chloro, bromo and iodo) cyano, nitro, amino, N,N-dimethylamino, phenyl, morpholinyl, or formyl group.

2. The compound of formula (I) according to claim 1, wherein A is —C(═O)NHOH, R₁ is hydrogen, R₂ is isobutyl, n-butyl, n-pentyl, benzyl or cyclopentylmethyl, R₃ is tert-butyl, iso-propyl, phenyl or benzyl, R₄ is hydrogen, n is 0 or 1, and R₅, R₆, R₇, R₈ and R₉ is independently hydrogen, methyl, fluoro, chloro, bromo, trifluoromethyl, methoxy, nitro, cyano or amino; or a pharmaceutically acceptable salts thereof.

3. The compound of formula (I) according to claim 1, wherein A is —N(CHO)OH, R₁ is hydrogen, R₂ is iso-butyl, n-butyl, n-pentyl, benzyl or cyclopentylmethyl, R₃ is tert-butyl, iso-propyl, phenyl or benzyl, R₄ is hydrogen, n is 0 or 1, and R₅, R₆, R₇, R₈ and R₉ is independently hydrogen, methyl, fluoro, chloro, bromo, trifluoromethyl, methoxy, nitro, cyano or amino; or a pharmaceutically acceptable salts thereof.

4. A process for preparing a compound of formula (I) or a pharmaceutically acceptable salt thereof, which comprises reacting a compound of formula (III) with hydroxylamine or an N- and/or O-protected hydroxylamine, and thereafter removing any N- or O-protecting groups: 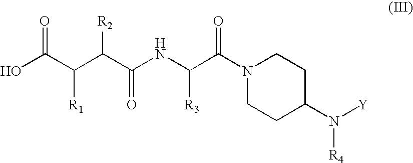
wherein, R₁, R₂, R₃, R₄ and Y are the same as defined in claim 1.

5. The method for preparing a compound of formula (III) according to claim 4 which process comprises reacting a compound of formula (IV) with a compound of formula (Va) (or Vb, or Vc) or salt thereof: 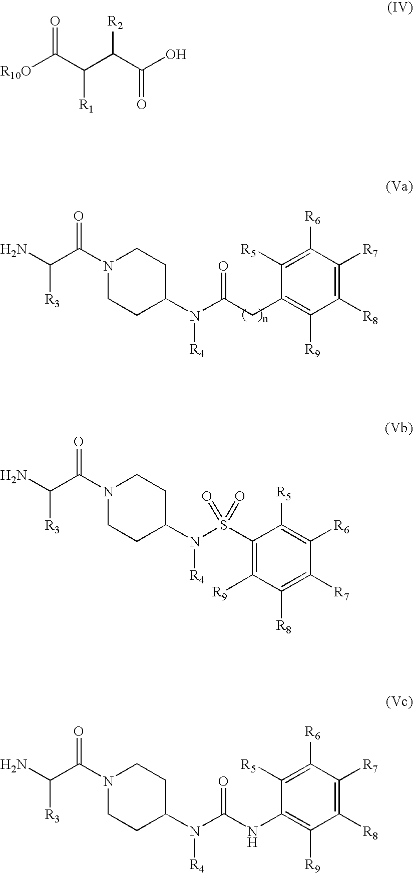
wherein, R₁, R₂, R₃, R₄, R₅, R₆, R₇, R₈, R₉ and n are the same as defined in claim 1 and R₁₀ is a hydroxy protecting group, such as methyl, ethyl, tert-butyl and benzyl.

6. A process for preparing a compound of formula (I) or a pharmaceutically acceptable salt thereof, which comprises reacting a compound of formula (VI) with a compound of formula (Va) (or Vb, or Vc) or salt thereof, and thereafter removing any N- or O-protecting groups: 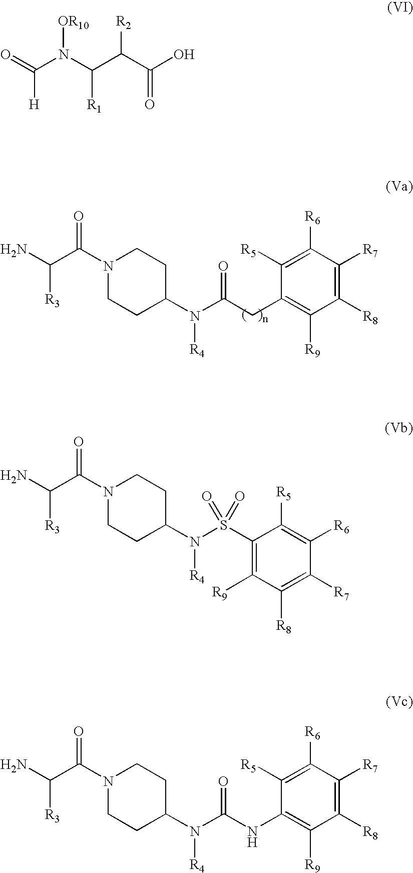
wherein R₁, R₂, R₃, R₄, R₅, R₆, R₇, R₈, R₉ and n are the same as defined in claim 1 and R₁₀ is a hydroxy protecting group, such as tert-butyl and benzyl.

7. An antibacterial composition comprising a therapeutically effective amount of the compound of formula (I) according to claim 1 or a pharmaceutically acceptable salt thereof.
